SEQUENCE LISTING (28 sequences)

SEQ ID NO: 1 - protein, 2048 aa, Homo sapiens
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 2 - protein, 540 aa, Homo sapiens
MALTRPVRLFSLVTRLLLAPRRGLTVRSPDEPLPVVRIPVALQRQLEQRQSRRRNLPRPVLVRPGPLLVSARRPELNQPARLTLGRWERAPLASQGWKSRRARRDHFSIERAQQEAPAVRKLSSKGSFADLGLEPRVLHALQEAAPEVVQPTTVQSSTIPSLLRGRHVVCAAETGSGKTLSYLLPLLQRLLGQPSLDSLPIPAPRGLVLVPSRELAQQVRAVAQPLGRSLGLLVRDLEGGHGMRRIRLQLSRQPSADVLVATPGALWKALKSRLISLEQLSFLVLDEADTLLDESFLELVDYILEKSHIAEGPADLEDPFNPKAQLVLVGATFPEGVGQLLNKVASPDAVTTITSSKLHCIMPHVKQTFLRLKGADKVAELVHILKHRDRAERTGPSGTVLVFCNSSSTVNWLGYILDDHKIQHLRLQGQMPALMRVGIFQSFQKSSRDILLCTDIASRGLDSTGVELVVNYDFPPTLQDYIHRAGRVGRVGSEVPGTVISFVTHPWDVSLVQKIELAARRRRSLPGLASSVKEPLPQAT

SEQ ID NO: 3 - protein, 356 aa, Homo sapiens
MDAGVTESGLNVTLTIRLLMHGKEVGSIIGKKGESVKRIREESGARINISEGNCPERIITLTGPTNAIFKAFAMIIDKLEEDINSSMTNSTAASRPPVTLRLVVPATQCGSLIGKGGCKIKEIRESTGAQVQVAGDMLPNSTERAITIAGVPQSVTECVKQICLVMLETLSQSPQGRVMTIPYQPMPASSPVICAGGQDRCSDAAGYPHATHDLEGPPLDAYSIQGQHTISPLDLAKLNQVARQQSHFAMMHGGTGFAGIDSSSPEVKGYWASLDASTQTTHELTIPNNLIGCIIGRQGANINEIRQMSGAQIKIANPVEGSSGRQVTITGSAASISLAQYLINARLSSEKGMGCS

SEQ ID NO: 4 - protein, 248 aa, Homo sapiens
MSGGGVIRGPAGNNDCRIYVGNLPPDIRTKDIEDVFYKYGAIRDIDLKNRRGGPPFAFVEFEDPRDAEDAVYGRDGYDYDGYRLRVEFPRSGRGTGRGGGGGGGGGAPRGRYGPPSRRSENRVVVSGLPPSGSWQDLKDHMREAGDVCYADVYRDGTGVVEFVRKEDMTYAVRKLDNTKFRSHEGETAYIRVKVDGPRSPSYGRSRSRSRSRSRSRSRSNSRSRSYSPRRSRGSPRYSPRHSRSRSRT

SEQ ID NO: 5 - protein, 502 aa, Homo sapiens
MDYDFKAKLAAERERVEDLFEYEGCKVGRGTYGHVYKARRKDGKDEKEYALKQIEGTGISMSACREIALLRELKHPNVIALQKVFLSHSDRKVWLLFDYAEHDLWHIIKFHRASKANKKPMQLPRSMVKSLLYQILDGIHYLHANWVLHRDLKPANILVMGEGPERGRVKIADMGFARLFNSPLKPLADLDPVVVTFWYRAPELLLGARHYTKAIDIWAIGCIFAELLTSEPIFHCRQEDIKTSNPFHHDQLDRIFSVMGFPADKDWEDIRKMPEYPTLQKDFRRTTYANSSLIKYMEKHKVKPDSKVFLLLQKLLTMDPTKRITSEQALQDPYFQEDPLPTLDVFAGCQIPYPKREFLNEDDPEEKGDKNQQQQQNQHQQPTAPPQQAAAPPQAPPPQQNSTQTNGTAGGAGAGVGGTGAGLQHSQDSSLNQVPPNKKPRLGPSGANSGGPVMPSDYQHSSSRLNYQSSVQGSSQSQSTLGYSSSSQQSSQYHPSHQAHRY

SEQ ID NO: 6 - protein, 480 aa, Homo sapiens
MAARCSTRWLLVVVGTPRLPAISGRGARPPREGVVGAWLSRKLSVPAFASSLTSCGPRALLTLRPGVSLTGTKHNPFICTASFHTSAPLAKEDYYQILGVPRNASQKEIKKAYYQLAKKYHPDTNKDDPKAKEKFSQLAEAYEVLSDEVKRKQYDAYGSAGFDPGASGSQHSYWKGGPTVDPEELFRKIFGEFSSSSFGDFQTVFDQPQEYFMELTFNQAAKGVNKEFTVNIMDTCERCNGKGNEPGTKVQHCHYCGGSGMETINTGPFVMRSTCRRCGGRGSIIISPCVVCRGAGQAKQKKRVMIPVPAGVEDGQTVRMPVGKREIFITFRVQKSPVFRRDGADIHSDLFISIAQALLGGTARAQGLYETINVTIPPGTQTDQKIRMGGKGIPRINSYGYGDHYIHIKIRVPKRLTSRQQSLILSYAEDETDVEGTVNGVTLTSSGGSTMDSSAGSKARREAGEDEEGFLSKLKKMFTS

SEQ ID NO: 7 - protein, 620 aa, Homo sapiens
MNLERLRKRVRQYLDQQQYQSALFWADKVASLSREEPQDIYWLAQCLYLTAQYHRAAHALRSRKLDKLYEACRYLAARCHYAAKEHQQALDVLDMEEPINKRLFEKYLKDESGFKDPSSDWEMSQSSIKSSICLLRGKIYDALDNRTLATYSYKEALKLDVYCFEAFDLLTSHHMLTAQEEKELLESLPLSKLCNEEQELLRFLFENKLKKYNKPSETVIPESVDGLQENLDVVVSLAERHYYNCDFKMCYKLTSVVMEKDPFHASCLPVHIGTLVELNKANELFYLSHKLVDLYPSNPVSWFAVGCYYLMVGHKNEHARRYLSKATTLEKTYGPAWIAYGHSFAVESEHDQAMAAYFTAAQLMKGCHLPMLYIGLEYGLTNNSKLAERFFSQALSIAPEDPFVMHEVGVVAFQNGEWKTAEKWFLDALEKIKAIGNEVTVDKWEPLLNNLGHVCRKLKKYAEALDYHRQALVLIPQNASTYSAIGYIHSLMGNFENAVDYFHTALGLRRDDTFSVTMLGHCIEMYIGDSEAYIGADIKDKLKCYDFDVHTMKTLKNIISPPWDFREFEVEKQTAEETGLTPLETSRKTPDSRPSLEETFEIEMNESDMMLETSMSDHST

SEQ ID NO: 8 - protein, 546 aa, Homo sapiens
MEQRRVTDFFARRRPGPPRIAPPKLACRTPSPARPALRAPASATSGSRKRARPPAAPGRDQARPPARRRLRLSVDEVSSPSTPEAPDIPACPSPGQKIKKSTPAAGQPPHLTSAQDQDTISELASCLQRARELGARVRALKASAQDAGESCTPEAEGRPEEPCGEKAPAYQRFHALAQPGLPGLVLPYKYQVLAEMFRSMDTIVGMLHNRSETPTFAKVQRGVQDMMRRRFEECNVGQIKTVYPASYRFRQERSVPTFKDGTRRSDYQLTIEPLLEQEADGAAPQLTASRLLQRRQIFSQKLVEHVKEHHKAFLASLSPAMVVPEDQLTRWHPRFNVDEVPDIEPAALPQPPATEKLTTAQEVLARARNLISPRMEKALSQLALRSAAPSSPGSPRPALPATPPATPPAASPSALKGVSQDLLERIRAKEAQKQLAQMTRCPEQEQRLQRLERLPELARVLRSVFVSERKPALSMEVACARMVGSCCTIMSPGEMEKHLLLLSELLPDWLSLHRIRTDTYVKLDKAADLAHITARLAHQTRAEEGL

SEQ ID NO: 9 - protein, 657 aa, Homo sapiens
MSQVLFHQLVPLQVKCKDCEERRVSIRMSIELQSVSNPVHRKDLVIRLTDDTDPFFLYNLVISEEDFQSLKFQQGLLVDFLAFPQKFIDLLQQCTQEHAKEIPRFLLQLVSPAAILDNSPAFLNVVETNPFKHLTHLSLKLLPGNDVEIKKFLAGCLKCSKEEKLSLMQSLDDATKQLDFTRKTLAEKKQELDKLRNEWASHTAALTNKHSQELTNEKEKALQAQVQYQQQHEQQKKDLEILHQQNIHQLQNRLSELEAANKDLTERKYKGDSTIRELKAKLSGVEEELQRTKQEVLSLRRENSTLDVECHEKEKHVNQLQTKVAVLEQEIKDKDQLVLRTKEAFDTIQEQKVVLEENGEKNQVQLGKLEATIKSLSAELLKANEIIKKLQGDLKTLMGKLKLKNTVTIQQEKLLAEKEEKLQKEQKELQDVGQSLRIKEQEVCKLQEQLEATVKKLEESKQLLKNNEKLITWLNKELNENQLVRKQDVLGPSTTPPAHSSSNTIRSGISPNLNVVDGRLTYPTCGIGYPVSSAFAFQNTFPHSISAKNTSHPGSGTKVQFNLQFTKPNASLGDVQSGATISMPCSTDKENGENVGLESKYLKKREDSIPLRGLSQNLFSNSDHQRDGTLGALHTSSKPTALPSASSAYFPGQLPNS

SEQ ID NO: 10 - protein, 197 aa, Homo sapiens
MAAFRDIEEVSQGLLSLLGANRAEAQQRRLLGRHEQVVERLLETQDGAEKQLREILTMEKEVAQSLLNAKEQVHQGGVELQQLEAGLQEAGEEDTRLKASLLQLTRELEELKEIEADLERQEKEVDEDTTVTIPSAVYVAQLYHQVSKIEWDYECEPGMVKGIHHGPSVAQPIHLDSTQLSRKFISDYLWSLVDTEW

SEQ ID NO: 11 - protein, 180 aa, Homo sapiens
MSVLRPLDKLPGLNTATILLVGTEDALLQQLADSMLKEDCASELKVHLAKSLPLPSSVNRPRIDLIVFVVNLHSKYSLQNTEESLRHVDASFFLGKVCFLATGAGRESHCSIHRHTVVKLAHTYQSPLLYCDLEVEGFRATMAQRLVRVLQICAGHVPGVSALNLLSLLRSSEGPSLEDL

SEQ ID NO: 12 - protein, 904 aa, Homo sapiens
MAESSESFTMASSPAQRRRGNDPLTSSPGRSSRRTDALTSSPGRDLPPFEDESEGLLGTEGPLEEEEDGEELIGDGMERDYRAIPELDAYEAEGLALDDEDVEELTASQREAAERAMRQRDREAGRGLGRMRRGLLYDSDEEDEERPARKRRQVERATEDGEEDEEMIESIENLEDLKGHSVREWVSMAGPRLEIHHRFKNFLRTHVDSHGHNVFKERISDMCKENRESLVVNYEDLAAREHVLAYFLPEAPAELLQIFDEAALEVVLAMYPKYDRITNHIHVRISHLPLVEELRSLRQLHLNQLIRTSGVVTSCTGVLPQLSMVKYNCNKCNFVLGPFCQSQNQEVKPGSCPECQSAGPFEVNMEETIYQNYQRIRIQESPGKVAAGRLPRSKDAILLADLVDSCKPGDEIELTGIYHNNYDGSLNTANGFPVFATVILANHVAKKDNKVAVGELTDEDVKMITSLSKDQQIGEKIFASIAPSIYGHEDIKRGLALALFGGEPKNPGGKHKVRGDINVLLCGDPGTAKSQFLKYIEKVSSRAIFTTGQGASAVGLTAYVQRHPVSREWTLEAGALVLADRGVCLIDEFDKMNDQDRTSIHEAMEQQSISISKAGIVTSLQARCTVIAAANPIGGRYDPSLTFSENVDLTEPIISRFDILCVVRDTVDPVQDEMLARFVVGSHVRHHPSNKEEEGLANGSAAEPAMPNTYGVEPLPQEVLKKYIIYAKERVHPKLNQMDQDKVAKMYSDLRKESMATGSIPITVRHIESMIRMAEAHARIHLRDYVIEDDVNMAIRVMLESFIDTQKFSVMRSMRKTFARYLSFRRDNNELLLFILKQLVAEQVTYQRNRFGAQQDTIEVPEKDLVDKARQINIHNLSAFYDSELFRMNKFSHDLKRKMILQQF

SEQ ID NO: 13 - protein, 617 aa, Homo sapiens
MKVSLGNGEMGVSAHLQPCKAGTTRFFTSNTHSSVVLQGFDQLRIEGLLCDVTLVPGDGDEIFPVHRAMMASASDYFKAMFTGGMKEQDLMCIKLHGVNKVGLKKIIDFIYTAKLSLNMDNLQDTLEAASFLQILPVLDFCKVFLISGVSLDNCVEVGRIANTYNLIEVDKYVNNFILKNFPALLSTGEFLKLPFERLAFVLSSNSLKHCTELELFKAACRWLRLEDPRMDYAAKLMKNIRFPLMTPQDLINYVQTVDFMRTDNTCVNLLLEASNYQMMPYMQPVMQSDRTAIRSDSTHLVTLGGVLRQQLVVSKELRMYDERAQEWRSLAPMDAPRYQHGIAVIGNFLYVVGGQSNYDTKGKTAVDTVFRFDPRYNKWMQVASLNEKRTFFHLSALKGHLYAVGGRSAAGELATVECYNPRMNEWSYVAKMSEPHYGHAGTVYGGLMYISGGITHDTFQNELMCFDPDTDKWMQKAPMTTVRGLHCMCTVGDKLYVIGGNHFRGTSDYDDVLSCEYYSPTLDQWTPIAAMLRGQSDVGVAVFENKIYVVGGYSWNNRCMVEIVQKYDPEKDEWHKVFDLPESLGGIRACTLTVFPPEENPGSPSRESPLSAPSDHS

SEQ ID NO: 14 - protein, 1332 aa, Homo sapiens
MESRDPAQPMSPGEATQSGARPADRYGLLKHSREFLDFFWDIAKPEQETRLAATEKLLEYLRGRPKGSEMKYALKRLITGLGVGRETARPCYSLALAQLLQSFEDLPLCSILQQIQEKYDLHQVKKAMLRPALFANLFGVLALFQSGRLVKDQEALMKSVKLLQALAQYQNHLQEQPRKALVDILSEVSKATLQEILPEVLKADLNIILSSPEQLELFLLAQQKVPSKLKKLVGSVNLFSDENVPRLVNVLKMAASSVKKDRKLPAIALDLLRLALKEDKFPRFWKEVVEQGLLKMQFWPASYLCFRLLGAALPLLTKEQLHLVMQGDVIRHYGEHVCTAKLPKQFKFAPEMDDYVGTFLEGCQDDPERQLAVLVAFSSVTNQGLPVTPTFWRVVRFLSPPALQGYVAWLRAMFLQPDLDSLVDFSTNNQKKAQDSSLHMPERAVFRLRKWIIFRLVSIVDSLHLEMEEALTEQVARFCLFHSFFVTKKPTSQIPETKHPFSFPLENQAREAVSSAFFSLLQTLSTQFKQAPGQTQGGQPWTYHLVQFADLLLNHSHNVTTVTPFTAQQRQAWDRMLQTLKELEAHSAEARAAAFQHLLLLVGIHLLKSPAESCDLLGDIQTCIRKSLGEKPRRSRTKTIDPQEPPWVEVLVEILLALLAQPSHLMRQVARSVFGHICSHLTPRALQLILDVLNPETSEDENDRVVVTDDSDERRLKGAEDKSEEGEDNRSSESEEESEGEESEEEERDGDVDQGFREQLMTVLQAGKALGGEDSENEEELGDEAMMALDQSLASLFAEQKLRIQARRDEKNKLQKEKALRRDFQIRVLDLVEVLVTKQPENALVLELLEPLLSIIRRSLRSSSSKQEQDLLHKTARIFTHHLCRARRYCHDLGERAGALHAQVERLVQQAGRQPDSPTALYHFNASLYLLRVLKGNTAEGCVHETQEKQKAGTDPSHMPTGPQAASCLDLNLVTRVYSTALSSFLTKRNSPLTVPMFLSLFSRHPVLCQSLLPILVQHITGPVRPRHQACLLLQKTLSMREVRSCFEDPEWKQLMGQVLAKVTENLRVLGEAQTKAQHQQALSSLELLNVLFRTCKHEKLTLDLTVLLGVLQGQQQSLQQGAHSTGSSRLHDLYWQAMKTLGVQRPKLEKKDAKEIPSATQSPISKKRKKKGFLPETKKRKKRKSEDGTPAEDGTPAATGGSQPPSMGRKKRNRTKAKVPAQANGTPTTKSPAPGAPTRSPSTPAKSPKLQKKNQKPSQVNGAPGSPTEPAGQKQHQKALPKKGVLGKSPLSALARKKARLSLVIRSPSLLQSGAKKKAQTLRFTISSSKK

SEQ ID NO: 15 - protein, 656 aa, Homo sapiens
MEELDGEPTVTLIPGVNSKKNQMYFDWGPGEMLVCETSFNKKEKSEMVPSCPFIYIIRKDVDVYSQILRKLFNESHGIFLGLQRIDEELTGKSRKSQLVRVSKNYRSVIRACMEEMHQVAIAAKDPANGRQFSSQVSILSAMELIWNLCEILFIEVAPAGPLLLHLLDWVRLHVCEVDSLSADVLGSENPSKHDSFWNLVTILVLQGRLDEARQMLSKEADASPASAGICRIMGDLMRTMPILSPGNTQTLTELELKWQHWHEECERYLQDSTFATSPHLESLLKIMLGDEAALLEQKELLSNWYHFLVTRLLYSNPTVKPIDLHYYAQSSLDLFLGGESSPEPLDNILLAAFEFDIHQVIKECSIALSNWWFVAHLTDLLDHCKLLQSHNLYFGSNMREFLLLEYASGLFAHPSLWQLGVDYFDYCPELGRVSLELHIERIPLNTEQKALKVLRICEQRQMTEQVRSICKILAMKAVRNNRLGSALSWSIRAKDAAFATLVSDRFLRDYCERGCFSDLDLIDNLGPAMMLSDRLTFLGKYREFHRMYGEKRFADAASLLLSLMTSRIAPRSFWMTLLTDALPLLEQKQVIFSAEQTYELMRCLEDLTSRRPVHGESDTEQLQDDDIETTKVEMLRLSLARNLARAIIREGSLEGS

SEQ ID NO: 16 - protein, 757 aa, Homo sapiens
MAAAEGPVGDGELWQTWLPNHVVFLRLREGLKNQSPTEAEKPASSSLPSSPPPQLLTRNVVFGLGGELFLWDGEDSSFLVVRLRGPSGGGEEPALSQYQRLLCINPPLFEIYQVLLSPTQHHVALIGIKGLMVLELPKRWGKNSEFEGGKSTVNCSTTPVAERFFTSSTSLTLKHAAWYPSEILDPHVVLLTSDNVIRIYSLREPQTPTNVIILSEAEEESLVLNKGRAYTASLGETAVAFDFGPLAAVPKTLFGQNGKDEVVAYPLYILYENGETFLTYISLLHSPGNIGKLLGPLPMHPAAEDNYGYDACAVLCLPCVPNILVIATESGMLYHCVVLEGEEEDDHTSEKSWDSRIDLIPSLYVFECVELELALKLASGEDDPFDSDFSCPVKLHRDPKCPSRYHCTHEAGVHSVGLTWIHKLHKFLGSDEEDKDSLQELSTEQKCFVEHILCTKPLPCRQPAPIRGFWIVPDILGPTMICITSTYECLIWPLLSTVHPASPPLLCTREDVEVAESPLRVLAETPDSFEKHIRSILQRSVANPAFLNCFTQLRKIKVETLFLRASEKDIAPPPEECLQLLSRATQVFREQYILKQDLAKEEIQRRVKLLCDQKKKQLEDLSYCREERKSLREMAERLADKYEEAKEKQEDIMNRMKKLLHSFHSELPVLSDSERDMKKELQLIPDQLRHLGNAIKQVTMKKDYQQQKMEKVLSLPKPTIILSAYQRKCIQSILKEEGEHIREMVKQINDIRNHVNF

SEQ ID NO: 17 - protein, 1232 aa, Homo sapiens
MKEEVKGIPVRVALRCRPLVPKEISEGCQMCLSFVPGEPQVVVGTDKSFTYDFVFDPSTEQEEVFNTAVAPLIKGVFKGYNATVLAYGQTGSGKTYSMGGAYTAEQENEPTVGVIPRVIQLLFKEIDKKSDFEFTLKVSYLEIYNEEILDLLCPSREKAQINIREDPKEGIKIVGLTEKTVLVALDTVSCLEQGNNSRTVASTAMNSQSSRSHAIFTISLEQRKKSDKNSSFRSKLHLVDLAGSERQKKTKAEGDRLKEGININRGLLCLGNVISALGDDKKGGFVPYRDSKLTRLLQDSLGGNSHTLMIACVSPADSNLEETLNTLRYADRARKIKNKPIVNIDPQTAELNHLKQQVQQLQVLLLQAHGGTLPGSITVEPSENLQSLMEKNQSLVEENEKLSRGLSEAAGQTAQMLERIILTEQANEKMNAKLEELRQHAACKLDLQKLVETLEDQELKENVEIICNLQQLITQLSDETVACMAAAIDTAVEQEAQVETSPETSRSSDAFTTQHALRQAQMSKELVELNKALALKEALARKMTQNDSQLQPIQYQYQDNIKELELEVINLQKEKEELVLELQTAKKDANQAKLSERRRKRLQELEGQIADLKKKLNEQSKLLKLKESTERTVSKLNQEIRMMKNQRVQLMRQMKEDAEKFRQWKQKKDKEVIQLKERDRKRQYELLKLERNFQKQSNVLRRKTEEAAAANKRLKDALQKQREVADKRKETQSRGMEGTAARVKNWLGNEIEVMVSTEEAKRHLNDLLEDRKILAQDVAQLKEKKESGENPPPKLRRRTFSLTEVRGQVSESEDSITKQIESLETEMEFRSAQIADLQQKLLDAESEDRPKQRWENIATILEAKCALKYLIGELVSSKIQVSKLESSLKQSKTSCADMQKMLFEERNHFAEIETELQAELVRMEQQHQEKVLYLLSQLQQSQMAEKQLEESVSEKEQQLLSTLKCQDEELEKMREVCEQNQQLLRENEIIKQKLTLLQVASRQKHLPKDTLLSPDSSFEYVPPKPKPSRVKEKFLEQSMDIEDLKYCSEHSVNEHEDGDGDDDEGDDEEWKPTKLVKVSRKNIQGCSCKGWCGNKQCGCRKQKSDCGVDCCCDPTKCRNRQQGKDSLGTVERTQDSEGSFKLEDPTEVTPGLSFFNPVCATPNSKILKEMCDVEQVLSKKTPPAPSPFDLPELKHVATEYQENKAPGKKKKRALASNTSFFSGCSPIEEEAH

SEQ ID NO: 18 - protein, 879 aa, Homo sapiens
MDPTAGSKKEPGGGAATEEGVNRIAVPKPPSIEEFSIVKPISRGAFGKVYLGQKGGKLYAVKVVKKADMINKNMTHQVQAERDALALSKSPFIVHLYYSLQSANNVYLVMEYLIGGDVKSLLHIYGYFDEEMAVKYISEVALALDYLHRHGIIHRDLKPDNMLISNEGHIKLTDFGLSKVTLNRDINMMDILTTPSMAKPRQDYSRTPGQVLSLISSLGFNTPIAEKNQDPANILSACLSETSQLSQGLVCPMSVDQKDTTPYSSKLLKSCLETVASNPGMPVKCLTSNLLQSRKRLATSSASSQSHTFISSVESECHSSPKWEKDCQESDEALGPTMMSWNAVEKLCAKSANAIETKGFNKKDLELALSPIHNSSALPTTGRSCVNLAKKCFSGEVSWEAVELDVNNINMDTDTSQLGFHQSNQWAVDSGGISEEHLGKRSLKRNFELVDSSPCKKIIQNKKTCVEYKHNEMTNCYTNQNTGLTVEVQDLKLSVHKSQQNDCANKENIVNSFTDKQQTPEKLPIPMIAKNLMCELDEDCEKNSKRDYLSSSFLCSDDDRASKNISMNSDSSFPGISIMESPLESQPLDSDRSIKESSFEESNIEDPLIVTPDCQEKTSPKGVENPAVQESNQKMLGPPLEVLKTLASKRNAVAFRSFNSHINASNNSEPSRMNMTSLDAMDISCAYSGSYPMAITPTQKRRSCMPHQQTPNQIKSGTPYRTPKSVRRGVAPVDDGRILGTPDYLAPELLLGRAHGPAVDWWALGVCLFEFLTGIPPFNDETPQQVFQNILKRDIPWPEGEEKLSDNAQSAVEILLTIDDTKRAGMKELKRHPLFSDVDWENLQHQTMPFIPQPDDETDTSYFEARNTAQHLTVSGFSL

SEQ ID NO: 19 - protein, 444 aa, Homo sapiens
  misc_feature (443)..(444): Xaa can be any naturally occurring amino acid
MRGAMELEPELLLQEARENVEAAQSYRRELGHRLEGLREARRQIKESASQTRDVLKQHFNDLKGTLGKLLDERLVTLLQEVDTIEQETIKPLDDCQKLIEHGVNTAEDLVREGEIAMLGGVGEENEKLWSFTKKASHIQLDSLPEVPLLVDVPCLSAQLDDSILNIVKDHIFKHGTVASRPPVQIEELIEKPGGIIVRWCKVDDDFTAQDYRLQFRKCTSNHFEDVYVGSETEFIVLHIDPNVDYQFRVCARGDGRQEWSPWSVPQIGHSTLVPHEWTAGFEGYSLSSRRNIALRNDSESSGVLYSRAPTYFCGQTLTFRVETVGQPDRRDSIGVCAEKQDGYDSLQRDQAVCISTNGAVFVNGKEMTNQLPAVTSGSTVTFDIEAVTLGTTSNNEGGHFKLRVTISSNNREVVFDWLLDQSCGSLYFGCSFFYPGWKVLVFXX

SEQ ID NO: 20 - protein, 290 aa, Homo sapiens
MAPKRQSPLPPQKKKPRPPPALGPEETSASAGLPKKGEKEQQEAIEHIDEVQNEIDRLNEQASEEILKVEQKYNKLRQPFFQKRSELIAKIPNFWVTTFVNHPQVSALLGEEDEEALHYLTRVEVTEFEDIKSGYRIDFYFDENPYFENKVLSKEFHLNESGDPSSKSTEIKWKSGKDLTKRSSQTQNKASRKRQHEEPESFFTWFTDHSDAGADELGEVIKDDIWPNPLQYYLVPDMDDEEGEGEEDDDDDEEEEGLEDIDEEGDEDEGEEDEDDDEGEEGEEDEGEDD

SEQ ID NO: 21 - protein, 395 aa, Homo sapiens
MSLVDLGKKLLEAARAGQDDEVRILMANGAPFTTDWLGTSPLHLAAQYGHYSTTEVLLRAGVSRDARTKVDRTPLHMAASEGHASIVEVLLKHGADVNAKDMLKMTALHWATEHNHQEVVELLIKYGADVHTQSKFCKTAFDISIDNGNEDLAEILQIAMQNQINTNPESPDTVTIHAATPQFIIGPGGVVNLTGLVSSENSSKATDETGVSAVQFGNSSTSVLATLAALAEASAPLSNSSETPVVATEEVVTAESVDGAIQQVVSSGGQQVITIVTDGIQLGNLHSIPTSGIGQPIIVTMPDGQQVLTVPATDIAEETVISEEPPAKRQCIEIIENRVESAEIEEREALQKQLDEANREAQKYRQQLLKKEQEAEAYRQKLEAMTRLQTNKEAV

SEQ ID NO: 22 - protein, 264 aa, Homo sapiens
MEAFLGSRSGLWAGGPAPGQFYRIPSTPDSFMDPASALYRGPITRTQNPMVTGTSVLGVKFEGGVVIAADMLGSYGSLARFRNISRIMRVNNSTMLGASGDYADFQYLKQVLGQMVIDEELLGDGHSYSPRAIHSWLTRAMYSRRSKMNPLWNTMVIGGYADGESFLGYVDMLGVAYEAPSLATGYGAYLAQPLLREVLEKQPVLSQTEARDLVERCMRVLYYRDARSYNRFQIATVTEKGVEIEGPLSTETNWDIAHMISGFE

SEQ ID NO: 23 - protein, 222 aa, Homo sapiens
MVKCCSAIGCASRCLPNSKLKGLTFHVFPTDENIKRKWVLAMKRLDVNAAGIWEPKKGDVLCSRHFKKTDFDRSAPNIKLKPGVIPSIFDSPYHLQGKREKLHCRKNFTLKTVPATNYNHHLVGASSCIEEFQSQFIFEHSYSVMDSPKKLKHKLDHVIGELEDTKESLRNVLDREKRFQKSLRKTIRELKDECLISQETANRLDTFCWDCCQESIEQDYIS

SEQ ID NO: 24 - protein, 177 aa, Homo sapiens
MEALILEPSLYTVKAILILDNDGDRLFAKYYDDTYPSVKEQKAFEKNIFNKTHRTDSEIALLEGLTVVYKSSIDLYFYVIGSSYENELMLMAVLNCLFDSLSQMLRKNVEKRALLENMEGLFLAVDEIVDGGVILESDPQQVVHRVALRGEDVPLTEQTVSQVLQSAKEQIKWSLLR

SEQ ID NO: 25 - protein, 625 aa, Homo sapiens
MNVTSIALRAETWLLAAWHVKVPPMWLEACINWIQEENNNVNLSQAQMNKQVFEQWLLTDLRDLEHPLLPDGILEIPKGELNGFYALQINSLVDVSQPAYSQIQKLRGKNTTNDLVTAEAQVTPKPWEAKPSRMLMLQLTDGIVQIQGMEYQPIPILHSDLPPGTKILIYGNISFRLGVLLLKPENVKVLGGEVDALLEEYAQEKVLARLIGEPDLVVSVIPNNSNENIPRVTDVLDPALGPSDEELLASLDENDELTANNDTSSERCFTTGSSSNTIPTRQSSFEPEFVISPRPKEEPSNLSIHVMDGELDDFSLEEALLLEETVQKEQMETKELQPLTFNRNADRSIERFSHNPNTTNNFSLTCKNGNNNWSEKNVSEQMTNEDKSFGCPSVRDQNRSIFSVHCNVPLAHDFTNKEKNLETDNKIKQTSSSDSHSLNNKILNREVVNYVQKRNSQISNENDCNLQSCSLRSSENSINLSIAMDLYSPPFVYLSVLMASKPKEVTTVKVKAFIVTLTGNLSSSGGIWSITAKVSDGTAYLDVDFVDEILTSLIGFSVPEMKQSKKDPLQYQKFLEGLQKCQRDLIDLCCLMTISFNPSLSKAMVLALQDVNMEHLENLKKRLNK

SEQ ID NO: 26 - protein, 1417 aa, Homo sapiens
MAAVPQNNLQEQLERHSARTLNNKLSLSKPKFSGFTFKKKTSSDNNVSVTNVSVAKTPVLRNKDVNVTEDFSFSEPLPNTTNQQRVKDFFKNAPAGQETQRGGSKSLLPDFLQTPKEVVCTTQNTPTVKKSRDTALKKLEFSSSPDSLSTINDWDDMDDFDTSETSKSFVTPPQSHFVRVSTAQKSKKGKRNFFKAQLYTTNTVKTDLPPPSSESEQIDLTEEQKDDSEWLSSDVICIDDGPIAEVHINEDAQESDSLKTHLEDERDNSEKKKNLEEAELHSTEKVPCIEFDDDDYDTDFVPPSPEEIISASSSSSKCLSTLKDLDTSDRKEDVLSTSKDLLSKPEKMSMQELNPETSTDCDARQISLQQQLIHVMEHICKLIDTIPDDKLKLLDCGNELLQQRNIRRKLLTEVDFNKSDASLLGSLWRYRPDSLDGPMEGDSCPTGNSMKELNFSHLPSNSVSPGDCLLTTTLGKTGFSATRKNLFERPLFNTHLQKSFVSSNWAETPRLGKKNESSYFPGNVLTSTAVKDQNKHTASINDLERETQPSYDIDNFDIDDFDDDDDWEDIMHNLAASKSSTAAYQPIKEGRPIKSVSERLSSAKTDCLPVSSTAQNINFSESIQNYTDKSAQNLASRNLKHERFQSLSFPHTKEMMKIFHKKFGLHNFRTNQLEAINAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLYSMVHYCENITECRRIQLLAYFGENGFNPDFCKKHPDVSCDNCCKTKDYKTRDVTDDVKSIVRFVQEHSSSQGMRNIKHVGPSGRFTMNMLVDIFLGSKSAKIQSGIFGKGSAYSRHNAERLFKKLILDKILDEDLYINANDQAIAYVMLGNKAQTVLNGNLKVDFMETENSSSVKKQKALVAKVSQREEMVKKCLGELTEVCKSLGKVFGVHYFNIFNTVTLKKLAESLSSDPEVLLQIDGVTEDKLEKYGAEVISVLQKYSEWTSPAEDSSPGISLSSSRGPGRSAAEELDEEIPVSSHYFASKTRNERKRKKMPASQRSKRRKTASSGSKAKGGSATCRKISSKTKSSSIIGSSSASHTSQATSGANSKLGIMAPPKPINRPFLKPSYAFS

SEQ ID NO: 27 - protein, 1001 aa, Homo sapiens
MIFPVARYALRWLRRPEDRAFSRAAMEMALRGVRKVLCVAEKNDAAKGIADLLSNGRMRRREGLSKFNKIYEFDYHLYGQNVTMVMTSVSGHLLAHDFQMQFRKWQSCNPLVLFEAEIEKYCPENFVDIKKTLERETRQCQALVIWTDCDREGENIGFEIIHVCKAVKPNLQVLRARFSEITPHAVRTACENLTEPDQRVSDAVDVRQELDLRIGAAFTRFQTLRLQRIFPEVLAEQLISYGSCQFPTLGFVVERFKAIQAFVPEIFHRIKVTHDHKDGIVEFNWKRHRLFNHTACLVLYQLCVEDPMATVVEVRSKPKSKWRPQALDTVELEKLASRKLRINAKETMRIAEKLYTQGYISYPRTETNIFPRDLNLTVLVEQQTPDPRWGAFAQSILERGGPTPRNGNKSDQAHPPIHPTKYTNNLQGDEQRLYEFIVRHFLACCSQDAQGQETTVEIDIAQERFVAHGLMILARNYLDVYPYDHWSDKILPVYEQGSHFQPSTVEMVDGETSPPKLLTEADLIALMEKHGIGTDATHAEHIETIKARMYVGLTPDKRFLPGHLGMGLVEGYDSMGYEMSKPDLRAELEADLKLICDGKKDKFVVLRQQVQKYKQVFIEAVAKAKKLDEALAQYFGNGTELAQQEDIYPAMPEPIRKCPQCNKDMVLKTKKNGGFYLSCMGFPECRSAVWLPDSVLEASRDSSVCPVCQPHPVYRLKLKFKRGSLPPTMPLEFVCCIGGCDDTLREILDLRFSGGPPRASQPSGRLQANQSLNRMDNSQHPQPADSRQTGSSKALAQTLPPPTAAGESNSVTCNCGQEAVLLTVRKEGPNRGRQFFKCNGGSCNFFLWADSPNPGAGGPPALAYRPLGASLGCPPGPGIHLGGFGNPGDGSGSGTSCLCSQPSVTRTVQKDGPNKGRQFHTCAKPREQQCGFFQWVDENTAPGTSGAPSWTGDRGRTLESEARSKRPRASSSDMGSTAKKPRKCSLCHQPGHTRPFCPQNR

SEQ ID NO: 28 - protein, 147 aa, Homo sapiens
MAAAADSFSGGPAGVRLPRSPPLKVLAEQLRRDAEGGPGAWRLSRAAAGRGPLDLAAVWMQGRVVMADRGEARLRDPSGDFSVRGLERVPRGRPCLVPGKYVMVMGVVQACSPEPCLQAVKMTDLSDNPIHESMWELEVEDLHRNIP

(19) United States
(12) Patent Application Publication
(10) Pub. No.: US 2019/0010517 A1
(43) Pub. Date: Jan. 10, 2019

(54) METHODS AND COMPOSITIONS FOR FACILITATING HOMOLOGOUS RECOMBINATION

(71) Applicant: The Board of Trustees of the Leland Stanford Junior University, Stanford, CA (US)

(72) Inventors: Mark A. Kay, Los Altos, CA (US); Gustavo De Alencastro, Palo Alto, CA (US)

(21) Appl. No.: 16/024,324

(22) Filed: Jun. 29, 2018

Related U.S. Application Data
(60) Provisional application No. 62/529,305, filed on Jul. 6, 2017.
(60) Provisional application No. 62/595,472, filed on Dec. 6, 2017.

(51) Int. Cl. C12N 15/90; C12N 15/864

(52) U.S. Cl. CPC C12N 15/902; C12N 5/0696; C12N 2310/20; C12N 15/8645

(57) ABSTRACT

Compositions and methods are provided for inducing/enhancing homologous recombination (HR) in a target cell (e.g., for editing a genome). In some embodiments, a subject method includes introducing into a cell: (a) a donor DNA (e.g., via a virus such as AAV); and (b) an agent that inhibits expression and/or function of an endogenous gene (e.g., an RNAi agent, a genome editing agent, a chemical inhibitor). In some cases, the endogenous gene is selected from: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RM12. In some embodiments, a subject method includes introducing into a target cell: (a) a donor DNA (e.g., via a virus such as AAV); and (b) an agent that inhibits centriole/centrosome assembly (e.g., an inhibitor of Polo-like kinase 4 (Plk4), centrinone, centrinone B, CFI-400945, R1530, and the like).

### CROSS-REFERENCE

This application claims the benefit of U.S. Provisional Patent Application Nos. 62/529,305 filed Jul. 6, 2017, and 62/595,472 filed Dec. 6, 2017, each of which application is incorporated herein by reference in its entirety.

### GOVERNMENT SUPPORT

This invention was made with Government support under contract HL064274 awarded by the National Institutes of Health. The Government has certain rights in the invention.

### INTRODUCTION

Gene therapy includes the introduction of nucleic acids (e.g., DNA) into cells in order to correct or prevent a pathological process. In general, the gene addition strategy has been used to transduce cells and introduce DNA (e.g., cDNA) that (i) encodes a desired gene product, and (ii) includes regulatory element(s) for appropriate gene expression. On the other hand, a gene repair strategy can be used to deliver tools for repairing a mutation in the genome, e.g., via homologous recombination (HR), which is usually an inefficient process in mammalian cells. The cell's ability to use an external DNA template (a “donor DNA”) for HR can be used to facilitate the insertion, deletion, or substitution of a desired sequence within the genome. HR can be invoked by providing a cell with a donor DNA with or without nucleases such as zinc finger nucleases (ZFNs), transcription activator-like effector nucleases (TALENs), and CRISPR/Cas nucleases (e.g., Cas9, Cpf1, CasX, CasY, and the like). Editing a cell's genome using HR has numerous research and clinical applications, just one of which is the correction of mutations that cause disease.

There is a need for compositions and methods that can facilitate (e.g., enhance the efficiency of) HR. This disclosure provides compositions and methods that address at least this need.

### SUMMARY

Through functional screening experiments, the inventors have discovered that reducing the expression and/or function of one or more genes can facilitate homologous recombination (HR) in a cell. This disclosure provides compositions and methods of inducing (e.g., enhancing) HR in a target cell. Such methods can also be referred to as methods of editing a genome because the provided methods can result in the incorporation of donor DNA sequence into the target cell's genomic DNA.

In some embodiments, a subject method (e.g., a method of inducing homologous recombination in a target cell, a method of editing a genome, a method of enhancing homologous recombination in a target cell, and the like) includes introducing into a target cell: (a) a donor DNA (in some cases introduced into the cell by a virus such as AAV); and (b) an agent that inhibits expression and/or function of an endogenous gene (e.g., an RNAi agent, a genome editing agent, and the like). In some cases, the endogenous gene is selected from: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and COPZ1. In some cases, the endogenous gene is selected from: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and COPZ1. In some cases, the endogenous gene is selected from: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2. In some cases, the endogenous gene is selected from: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2. In some cases, the endogenous gene is selected from: FANCM, DDX28, PCBP1, SFRS1/SRSF1, CDK19, DNAJA3/TID1, CDC16, and CDT1. In some cases, the endogenous gene is selected from: SASS6, SPC24, and CENPM. In some cases, the endogenous gene is selected from: FANCM, RMI1, BLM, TOP3A, and RMI2. In some cases, the endogenous gene is selected from: FANCM and RMI1. In some cases, the endogenous gene is FANCM.

In some cases, an agent (or agents) are used that inhibit expression and/or function of two or more endogenous genes (e.g., an RNAi agent(s), a genome editing agent(s), and the like). In some such cases, one of the endogenous genes is FANCM and another encodes a member of the BTR complex (e.g., RMI1 or BLM). In some cases, the two or more endogenous genes includes FANCM and RMI1. In some cases, the two or more endogenous genes includes FANCM and BLM.

The discovery by the inventors that reduced expression/function of genes/proteins such as SASS6, SPC24, and CENPM leads to enhanced homologous recombination indicated that inhibition of centriole/centrosome assembly can also enhance homologous recombination. Thus, in some embodiments, a subject method (e.g., a method of inducing homologous recombination in a target cell, a method of editing a genome, a method of enhancing homologous recombination in a target cell, and the like) includes introducing into a target cell: (a) a donor DNA (in some cases introduced into the cell by a virus such as AAV); and (b) an agent that inhibits centriole/centrosome assembly (e.g., an inhibitor of Polo-like kinase 4 (Plk4), centrinone, centrinone B, CFI-400945, R1530, and the like).

In some cases a subject method includes cleavage of the target cell's genomic DNA with a gene editing endonuclease (e.g., a CRISPR/Cas programmable endonuclease, a zinc finger nuclease (ZFN), a TALE nuclease (TALEN), and the like) at a locus with homology to the donor DNA, prior to incorporation of the sequence of the donor DNA. In some cases where the genome is cleaved prior to HR, the subject methods can reduce the chances of undesired non-homologous end-joining (NHEJ), which is a DNA repair pathway the cell can use instead of HR. In some cases a subject method does not include a step of cleaving the target cell's genomic DNA prior to incorporation of the sequence of the donor DNA (e.g., the target cell's genome is not cleaved with a gene editing endonuclease at a locus with homology to the donor DNA prior to incorporation of the sequence of the donor DNA).

Also provided are genetically modified cells (and non-human animals having such cells) with enhanced HR and methods of generating such cells and/or non-human animals. Genetically modified cells with enhanced HR can have a reduced level of wild type protein produced from the endogenous genomic locus of one or more target genes selected from: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and COPZ1 (e.g., due to an altered nucleotide sequence at a promoter and/or enhancer that reduces transcription, due to an altered protein coding nucleotide sequence that encodes a loss of function allele such as a null allele or an allele with partially reduced function, due to an RNAi agent that specifically targets expression of the targeted gene, etc.). In some cases, a genetically modified cell with enhanced HR has a reduced level of wild type protein produced from the endogenous genomic locus of one or more target genes selected from: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and COPZ1. In some cases, a genetically modified cell with enhanced HR has a reduced level of wild type protein produced from the endogenous genomic locus of one or more target genes selected from: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2. In some cases, a genetically modified cell with enhanced HR has a reduced level of wild type protein produced from the endogenous genomic locus of one or more target genes selected from: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2. In some cases, a genetically modified cell with enhanced HR has a reduced level of wild type protein produced from the endogenous genomic locus of one or more target genes selected from: FANCM, DDX28, PCBP1, SFRS1/SRSF1, CDK19, DNAJA3/TID1, CDC16, and CDT1. In some cases, a genetically modified cell with enhanced HR has a reduced level of wild type protein produced from the endogenous genomic locus of one or more target genes selected from: SASS6, SPC24, and CENPM. In some cases, a genetically modified cell with enhanced HR has a reduced level of wild type protein produced from the endogenous genomic locus of one or more target genes selected from: FANCM, RMI1, BLM, TOP3A, and RMI2. In some cases, a genetically modified cell with enhanced HR has a reduced level of wild type protein produced from the endogenous FANCM and/or RMI1 locus. In some cases, a genetically modified cell with enhanced HR has a reduced level of wild type protein produced from the endogenous FANCM locus.

Also provided are kits for performing the methods of the disclosure (e.g., a kit for genome editing, a kit for HR, and the like).

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. GFP expression in wild type HAP1 cells transduced with AAV-DJ and expressing green fluorescent protein (GFP) under the control of the CAG promoter. Cells were plated 24 hours before being transduced with three different multiplicity of infection (MOIs): 1,000; 10,000; and 100,000. GFP expression was analyzed 48 hours post-transduction by FACs analysis.

FIG. 2. Schematic of AAV-GAPDH-2A-BLAS targeting construct. The AAV-DJ capsid packages the genomes as designated. The resulting recombination event results in a single mRNA that makes two proteins: one encoded by the endogenous genomic locus (e.g. GAPDH) and the other was the transgene carried in the vector (Blasticidin in this case). Expression is based on the endogenous cellular promoter. The chance that a random integration will result in activation of the neighboring DNA is greatly reduced because there is no promoter in the introduced vector.

FIG. 3. Scheme of protein associations between known HR and NHEJ associated proteins and the enriched proteins found in the HAP1 screening. This interaction network was generated using the String software (“htt” followed by “tp://” followed by “string-db.” followed by “org”), which infers protein associations based on shared functions, known interactions (curated databases and experimentally determined), predicted interactions (gene neighborhood, gene fusions and gene co-occurrence), and others (text mining, co-expression and protein homology). However, their association does not necessarily mean they physically interact with one another.

FIG. 4. Schematic of AAV-GAPDH-2A-GFP targeting construct. The AAV-DJ capsid packages the genomes as designated. The resulting recombination event results in a single mRNA that makes two proteins: the one encoded by the endogenous genomic locus (e.g. GAPDH) and the other was the transgene carried in the vector (GFP in this case). Expression is based on the endogenous cellular promoter. The chance that a random integration will result in activation of the neighboring DNA is greatly reduced because there is no promoter in the introduced vector.

FIG. 5. GFP expression in wild type and three FANCM knock out HAP1 clones (B12, C1 and C3) transduced with AAV-GAPDH-2A-GFP. Cells were plated in duplicate 24 hours before being transduced with an MOI: 5,000. GFP expression was analyzed 48 hours post-transduction by FACs analysis. The letters A and B represent the duplicates.

FIG. 6. GFP expression in wild type and three CDK19 knock out HAP1 clones (A1, A3 and C5) transduced with AAV-GAPDH-2A-GFP. Cells were plated in duplicate 24 hours before being transduced with an MOI: 5,000. GFP expression was analyzed 48 hours post-transduction by FACs analysis. The letters A and B represent the duplicates.

FIG. 7. Wild type protein sequences corresponding to the following genes: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI, BLM, TOP3A, and RMI2.

FIG. 8. GFP expression in wild type and FANCM knock out HeLa cells transduced with AAV-GAPDH-2A-GFP. Cells were plated in triplicate 24 hours before being transduced with an MOI: 51,000. GFP expression was analyzed 48 hours post-transduction by FACs analysis. The letters A, B and C represent the triplicates.

FIG. 9. AAV injections for analysis of AAV-HR in vivo. Tail-vein injection of an AAV expressing sgRNAs (CRISPR/Cas genome editing agents) against FANCM (blue line) and saline (red line) is followed by infusion of the AAV8-Albumin-2A-hF9 targeting vector at various times. The relative levels of HR are determined by plasma human factor IX measurements.

FIG. 10. Schematic representation of FANCM protein interactions. When two replication forks have collided, FANCM binds to inter-strand crosslink DNA and recruits and interacts with two complexes of DNA repair: the FA core and the Bloom dissolvasome (BTR complex). The interaction between Bloom dissolvasome complex and FANCM occurs via the RMI subcomplex, which physically anchors the Bloom dissolvasome to FANCM by binding to a 34 amino acid motif in FANCM called MM2.

FIG. 11. GFP expression in wild type and RMI1 knock out HeLa cells transduced with AAV-GAPDH-2A-GFP. Cells were plated in triplicate 24 hours before being transduced with an MOI: 37,000. GFP expression was analyzed 48 hours post-transduction by FACs analysis. The letters A, B and C represent triplicates.

FIG. 12. GFP expression in wild type HeLa cells transfected with different combinations of siRNAs followed by transduction with AAV-GAPDH-2A-GFP.

### DETAILED DESCRIPTION

As summarized above, compositions and methods for are provided for facilitating (e.g., inducing, enhancing, etc.) homologous recombination (HR) in a target cell. Also provided are genetically modified cells (and non-human animals having such cells) with enhanced HR and methods of generating such cells and/or non-human animals.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is human.

The terms “specific binding,” “specifically binds,” and the like, refer to non-covalent or covalent preferential binding to a molecule relative to other molecules or moieties in a solution or reaction mixture (e.g., an antibody can specifically bind to an antigen such as a particular protein relative to other available proteins). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁶ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The terms “co-administration”, “co-administer”, and “in combination with” include the administration of two or more agents either simultaneously, concurrently or sequentially within no specific time limits. In some embodiments, the agents are present in the cell or in the subject's body at the same time or exert their biological or therapeutic effect at the same time. In some embodiments, the therapeutic agents are in the same composition or unit dosage form. In other embodiments, the therapeutic agents are in separate compositions or unit dosage forms. In certain embodiments, a first agent (e.g., a gene editing agent, an agent that inhibits expression and/or function of a particular gene) can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), or concomitantly with the administration of a second agent (e.g., a donor polynucleotide).

“Dosage unit” refers to physically discrete units suited as unitary dosages for a particular individual to be treated (e.g., a dosage unit of an agent that will increase the efficiency of HR of a target cell). Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as those for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease or condition, is sufficient to effect treatment for that disease or condition.

The terms “treatment”, “treating”, “treat” and the like are used herein to generally refer to obtaining a desired pharmacologic and/or physiologic effect (e.g., increased/enhanced HR).

### Methods and Compositions

As summarized above and as described in the examples below, the inventors have discovered that reducing the expression and/or function of one or more genes can increase the efficiency of homologous recombination (HR) in a cell. Thus, this disclosure provides compositions and methods of facilitating HR in a target cell. The disclosure provides compositions and methods of inducing (e.g., enhancing) HR in a target cell. Such methods can also be referred to as methods of editing a genome because the provided methods can result in the incorporation of donor DNA sequence (i.e., sequence of a donor DNA) into the target cell's genomic DNA. Methods of the disclosure (e.g., methods of facilitating homologous recombination in a target cell, methods of inducing homologous recombination in a target cell, methods of editing a genome, methods of enhancing homologous recombination in a target cell, and the like) can include introducing into a target cell: (a) a donor DNA; and (b) an agent that inhibits expression and/or function of an endogenous gene (see, e.g., description below, e.g., Table 1).

### Increasing HR

Homologous recombination (HR) is by nature an inefficient process and methods of the disclosure can increase this efficiency. In general, increasing HR refers to increasing the percent of cells in target population of cells that undergo HR after introduction of a donor DNA. For example, increased HR mediated by an agent (e.g., an agent such as an RNAi agent that inhibits expression and/or function of an endogenous gene; an agent that inhibits centriole assembly; and the like) can be measured by showing that more HR is achieved (a higher fraction of cells in the target cell population incorporate donor DNA sequence) when an agent (plus a donor DNA) is introduced to the cells than when the agent is not introduced (e.g., when the donor DNA is introduced in the absence of the agent).

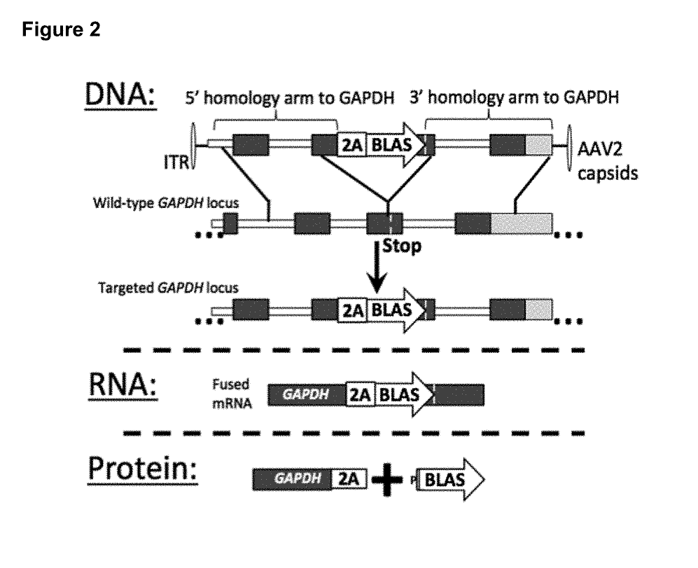

HR can be measured using any convenient method and a number of methods will be available to one of ordinary skill in the art. For example, the donor DNA sequence to be incorporated can include an identifiable marker sequence (e.g., can encode a marker protein such as a drug selectable marker, a fluorescent protein, and the like), an introduced restriction site (that can be assayed, e.g., by amplifying the targeted region and contacting the amplified DNA with an appropriate restriction enzyme), or any identifiable sequence that differs from the endogenous sequence (e.g., the donor DNA sequence can be detected by amplifying the targeted region and sequencing the amplified product). See, for example, FIGS. 2-5 and the Examples section below.

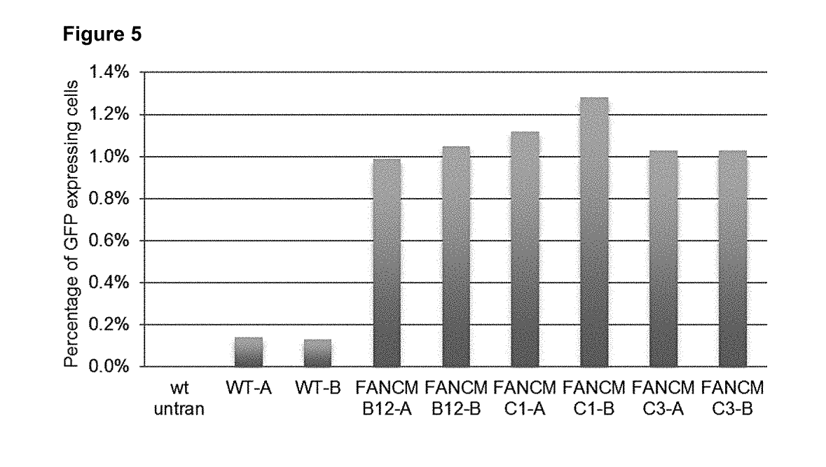

In some cases, HR is increased by 20% or more (e.g., 30% or more, 40% or more, 50% or more, 60% or more, 70% or more, 80% or more, 90% or more, 100% or more, 150% or more, 200% or more, etc.) in the presence of a subject agent (an agent that inhibits expression and/or function of an endogenous gene, e.g., FANCM) as compared to in the absence of the agent. In some cases, the ratio of the frequency of HR (% of cells in a cell population that undergo HR) in the presence of a subject agent (e.g., when the agent is introduced into the cells) compared to the frequency of HR in the absence of the agent is 1.2 or greater (e.g., 1.5 or greater, 2 or greater, 3 or greater, 5 or greater, 10 or greater, 20 or greater, etc.)(FIG. 5 demonstrates a ratio of roughly 5). In other words, in some cases the introduction of a subject agent (an agent that inhibits expression and/or function of an endogenous gene, e.g., FANCM) results in an improvement (increase) of HR frequency (compared to HR frequency in the absence of the agent) of 1.2-fold or more (e.g., 1.5-fold or more, 2-fold or more, 3-fold or more, 5-fold or more, 10-fold or more, 20-fold or more, etc.) (FIG. 5 demonstrates an improvement of roughly 10-fold). In some cases, the improvement is 5-fold or more.

In some cases where the genome is cleaved prior to HR, the subject methods can reduce the chances of undesired non-homologous end-joining (NHEJ), which is a DNA repair pathway the cell can use instead of HR. Thus, in some cases a subject method results in an increased ratio of HR:NHEJ when a subject agent is added (in addition to the donor polynucleotide) compared to when the agent is not added (e.g., when an agent such as an RNAi agent that inhibits expression and/or function of an endogenous gene is introduced in addition to a donor DNA compared to when a donor DNA is introduced in the absence of the agent).

### Endogenous Target Genes for Facilitating/Inducing HR

As noted above, an agent used in the subject methods is an agent that inhibits expression and/or function of an endogenous gene. The inventors have performed experiments showing that HR frequency increases when the wild type function of an endogenous gene is reduced, when the endogenous gene is at least one of the following genes: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and COPZ1 (see Table 1). The inventors have performed experiments showing that HR frequency increases when the wild type function of an endogenous gene is reduced, when the endogenous gene is at least one of the following genes: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2 (see Table 1). Thus, in some embodiments (e.g., in methods of the disclosure) the endogenous gene is selected from the group consisting of: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and COPZ1. In some cases (e.g., in methods of the disclosure) the endogenous gene is selected from the group consisting of: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2. In some cases (e.g., in methods of the disclosure) the endogenous gene is selected from the group consisting of: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and COPZ1. In some cases (e.g., in methods of the disclosure) the endogenous gene is selected from the group consisting of: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2. In some cases (e.g., in methods of the disclosure) the endogenous gene is selected from the group consisting of: FANCM, DDX28, PCBP1, SFRS1/SRSF1, CDK19, DNAJA3/TID1, CDC16, and CDT1. In some cases (e.g., in methods of the disclosure) the endogenous gene is selected from the group consisting of: SASS6, SPC24, and CENPM. In some cases (e.g., in methods of the disclosure) the endogenous gene is selected from the group consisting of: FANCM, RMI1, BLM, TOP3A, and RMI2. In some cases (e.g., in methods of the disclosure) the endogenous gene is selected from the group consisting of: FANCM and RMI1. In some cases (e.g., in methods of the disclosure) the endogenous gene is FANCM. In some cases (e.g., in methods of the disclosure) the endogenous gene is RMI1. In some cases (e.g., in methods of the disclosure) the endogenous gene is BLM.

The inventors have performed experiments (see, e.g., Example 4) showing that HR frequency increases when the wild type function of two or more endogenous genes is reduced. Thus, in some cases, an agent (or agents) are used that inhibit expression and/or function of two or more endogenous genes (e.g., an RNAi agent(s), a genome editing agent(s), and the like). In some such cases, one of the endogenous genes is FANCM and another encodes a member of the BTR complex (e.g., RMI1 or BLM). In some cases, the two or more endogenous genes includes FANCM and RMI1. In some cases, the two or more endogenous genes includes FANCM and BLM.

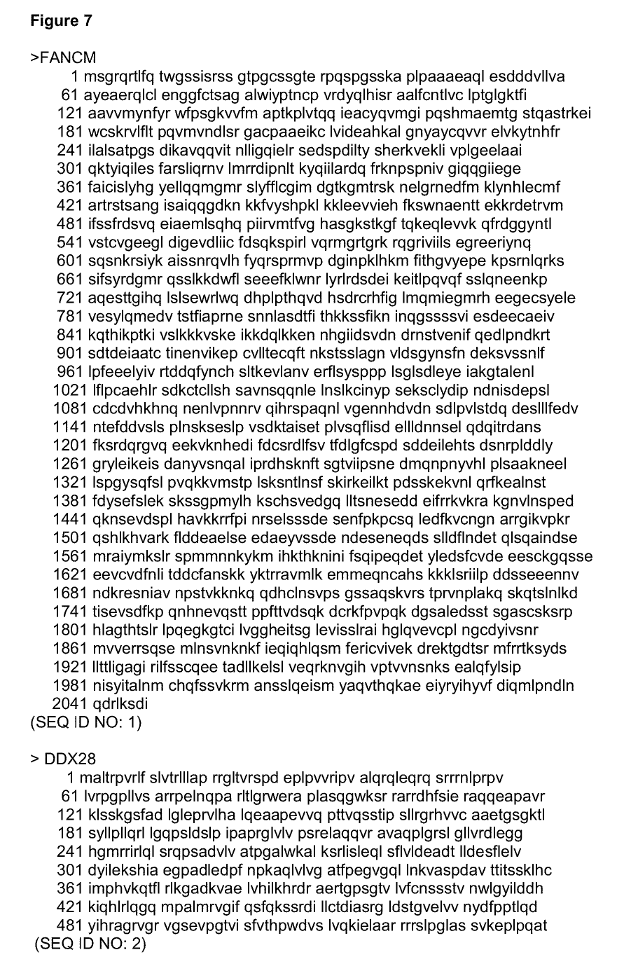

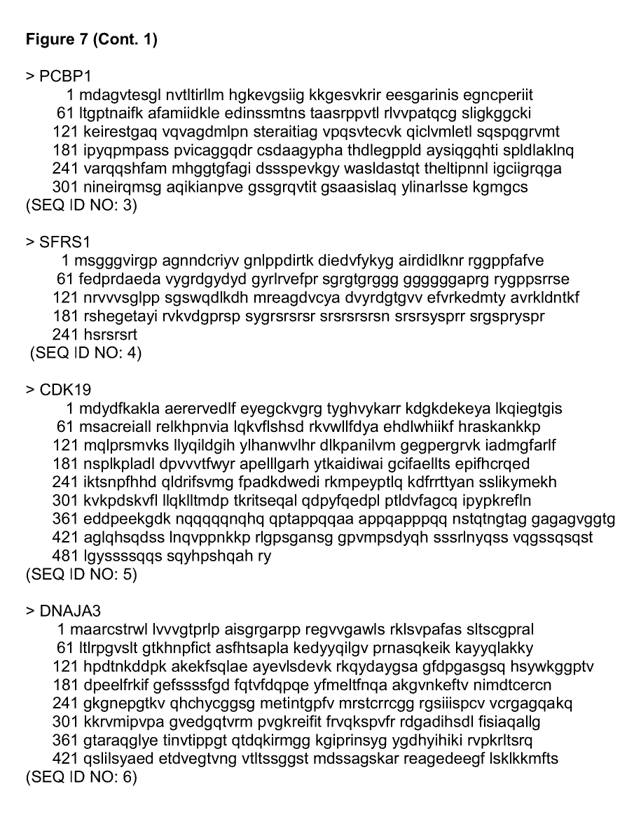

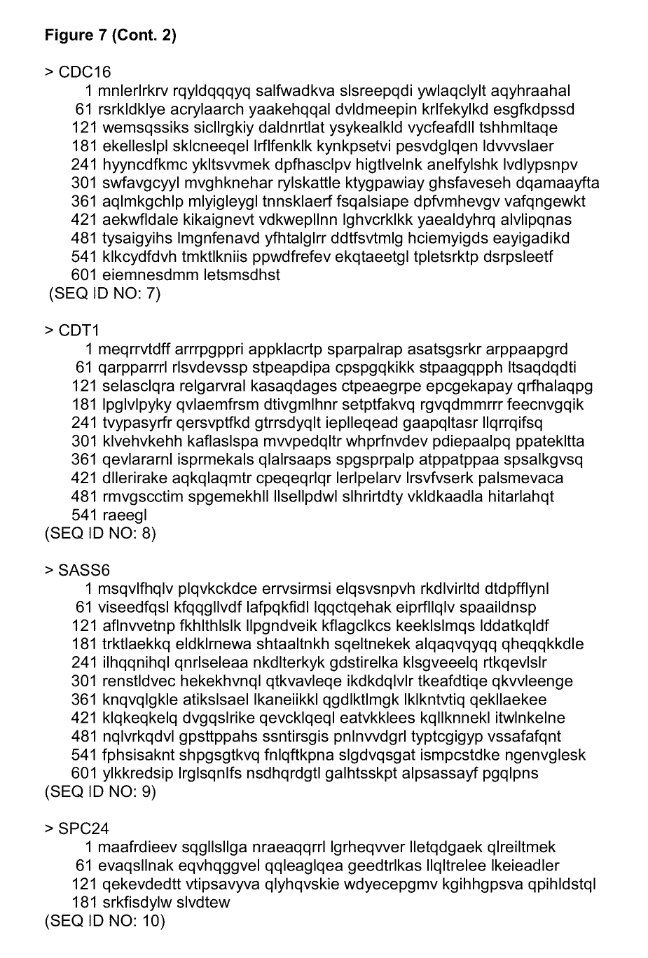

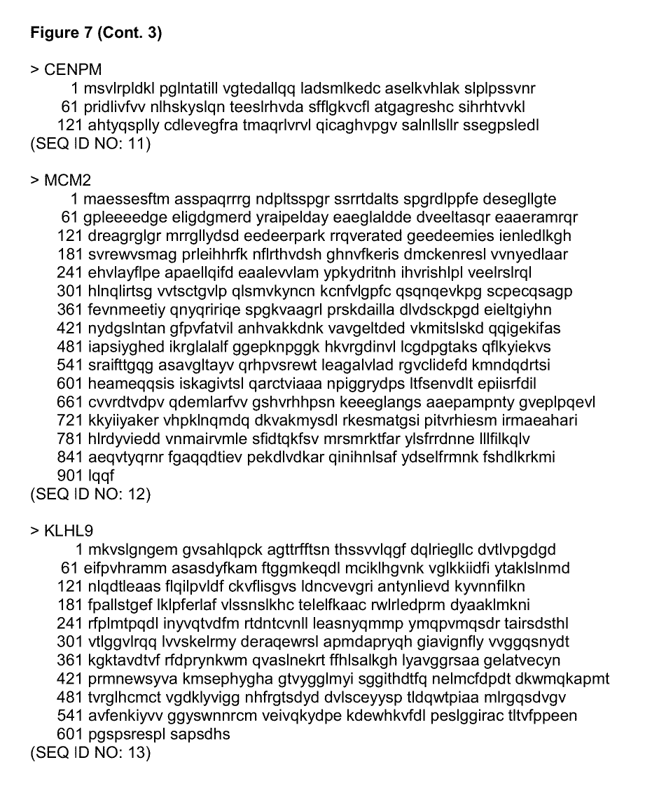

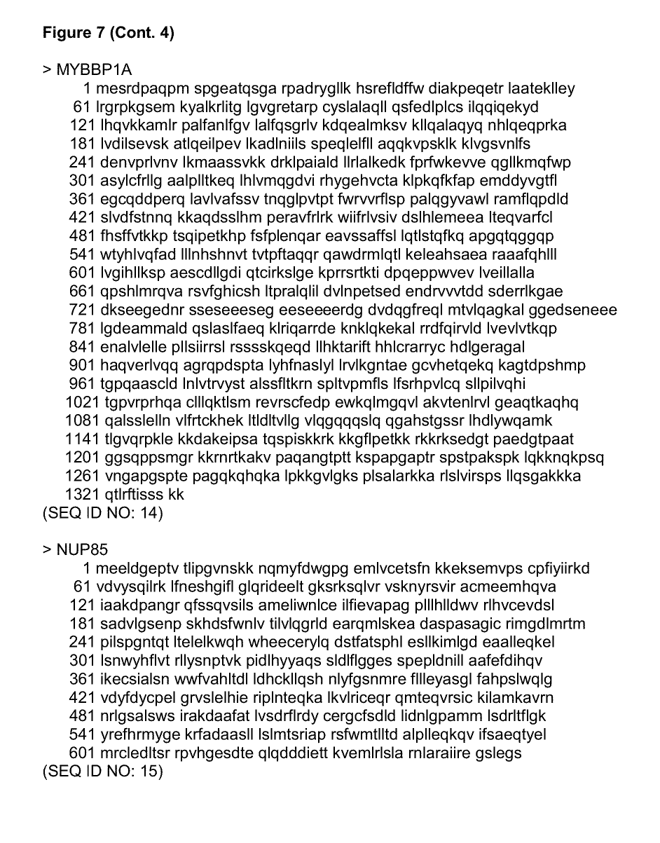

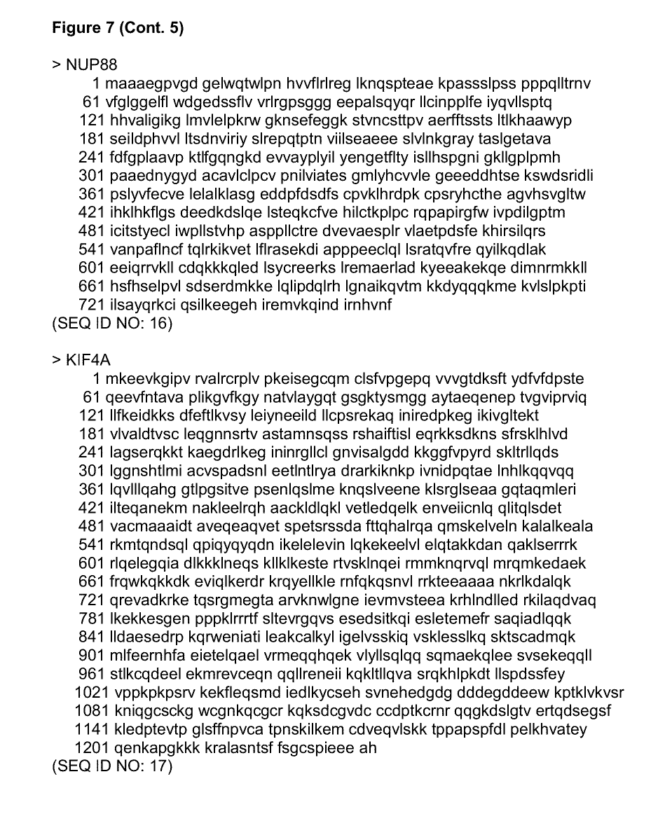

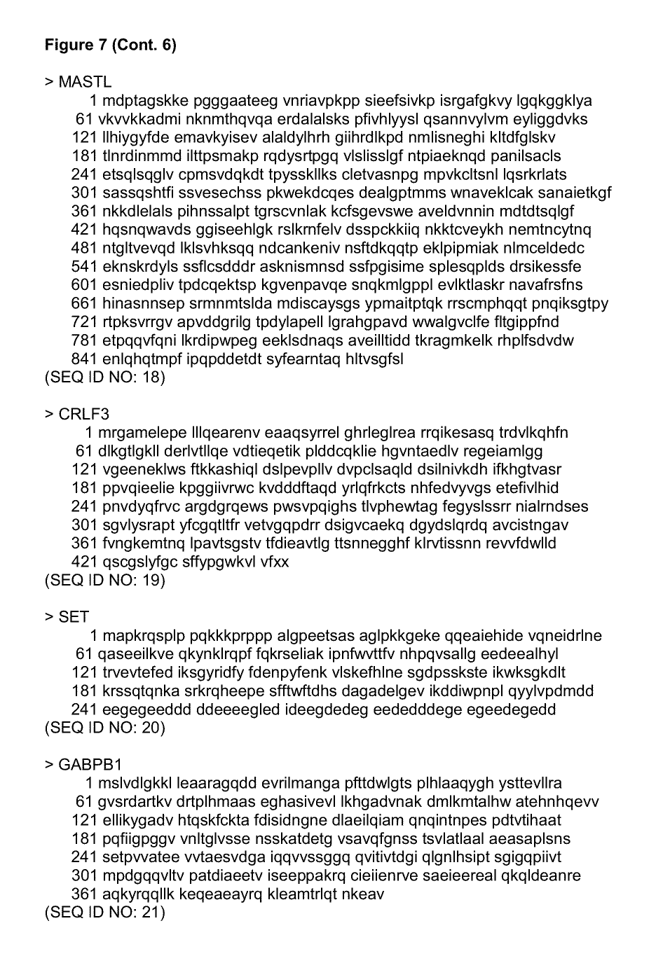

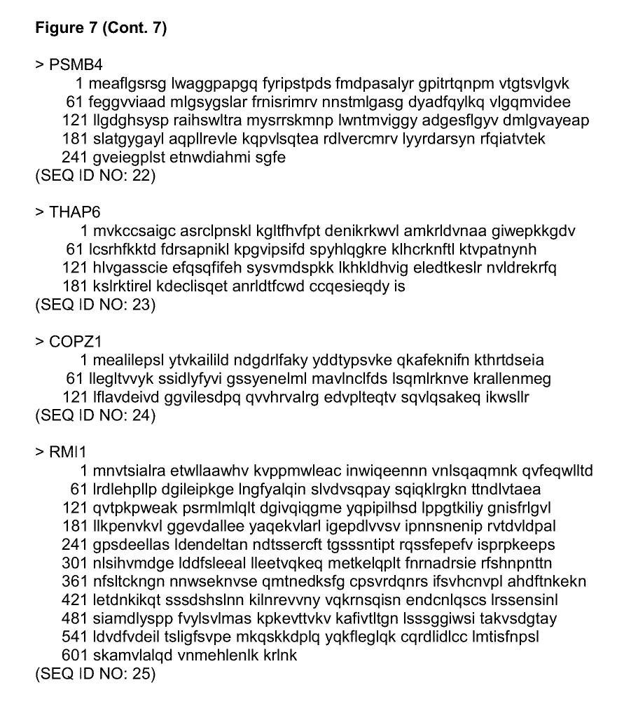

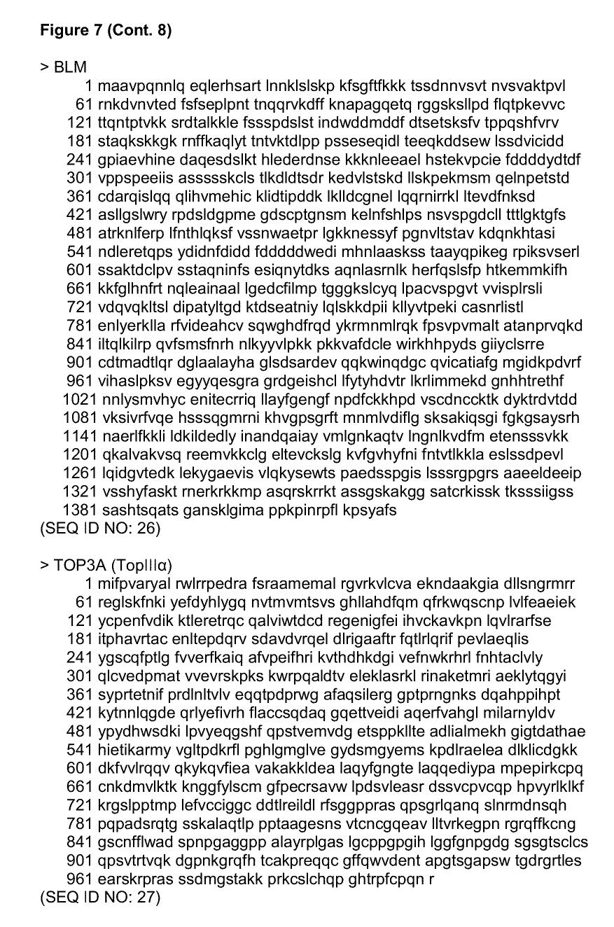

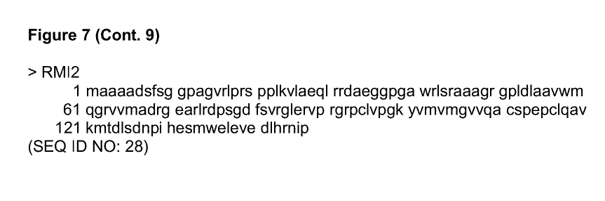

TABLE 1
Target genes of the disclosure. The SEQ ID NO. provided for
each gene is the corresponding protein. In cases where more
than one isoform of the protein exists, only one isoform is
listed, but others are available at NCBI (e.g., see the associated
NCBI gene ID number) (see, e.g., FIG. 7).

| Human gene | | NCBI | SEQ ID |
|---|---|---|---|
| name | Also known as | Gene ID | NO. |
| FANCM | Fanconi anemia | 57697 | 1 |
| | complementation group M | | |
| DDX28 | DEAD-box helicase 28 | 55794 | 2 |
| PCBP1 | poly(rC) binding protein 1 | 5093 | 3 |
| SFRS1 | serine and arginine rich | 6426 | 4 |
| (SFRS1/SRSF1) | splicing factor 1 | | |
| CDK19 | cyclin dependent kinase 19 | 23097 | 5 |
| DNAJA3 | TID1; DnaJ heat shock | 9093 | 6 |
| (DNAJA3/TID1) | protein family (Hsp40) | | |
| | member A3 | | |
| CDC16 | cell division cycle 16 | 8881 | 7 |
| CDT1 | chromatin licensing and DNA | 81620 | 8 |
| | replication factor 1 | | |
| SASS6 | SAS-6 centriolar assembly | 163786 | 9 |
| | protein; SAS6; SAS-6; | | |
| | MCPH14 | | |
| SPC24 | SPC24, NDC80 kinetochore | 147841 | 10 |
| | complex component, SPBC24 | | |
| CENPM | centromere protein M, | 79019 | 11 |
| | C22orf18, CENP-M, PANE1 | | |
| MCM2 | minichromosome | 4171 | 12 |
| | maintenance complex | | |
| | component 2, BM28, CCNL1, | | |
| | CDCL1, D3S3194, DFNA70, | | |
| | MITOTIN, cdc19 | | |
| KLHL9 | kelch like family member 9 | 55958 | 13 |
| MYBBP1A | MYB binding protein 1a, | 10514 | 14 |
| | P160, PAP2, Pol5 | | |
| NUP85 | nucleoporin 85, FROUNT, | 79902 | 15 |
| | Nup75 | | |
| NUP88 | nucleoporin 88 | 4927 | 16 |
| KIF4A | kinesin family member 4A, | 24137 | 17 |
| | KIF4, KIF4G1, MRX100 | | |
| MASTL | microtubule associated | 84930 | 18 |
| | serine/threonine kinase like, | | |
| | GREATWALL, GW, GWL, | | |
| | MAST-L, THC2 | | |
| CRLF3 | cytokine receptor like factor 3, | 51379 | 19 |
| | CREME-9, CREME9, | | |
| | CRLM9, CYTOR4, FRWS, | | |
| | p48.2 | | |
| SET | SET nuclear proto-oncogene, | 6418 | 20 |
| | 2PP2A, I2PP2A, IGAAD, | | |
| | IPP2A2, PHAPII, TAF-I, TAF- | | |
| | IBETA | | |
| GABPB1 | GA binding protein | 2553 | 21 |
| | transcription factor beta | | |
| | subunit 1, BABPB2, E4TF1, | | |
| | E4TF1-47, E4TF1-53, | | |
| | E4TF1B, GABPB, GABPB-1, | | |
| | GABPB2, NRF2B1, NRF2B2 | | |
| PSMB4 | proteasome subunit beta 4, | 5692 | 22 |
| | HN3, HsN3, PROS-26, | | |
| | PROS26 | | |
| THAP6 | THAP domain containing 6, | 152815 | 23 |
| COPZ1 | coatomer protein complex | 22818 | 24 |
| | subunit zeta 1, CGI-120, | | |
| | COPZ, HSPC181, zeta-COP, | | |
| | zeta1-COP | | |
| RMI1 | RecQ-mediated genome | 80010 | 25 |
| | instability 1, BLAP75, | | |
| | C9orf76, FAAP75 | | |
| BLM | Bloom DNA helicase, Bloom | 641 | 26 |
| | syndrome RecQ like | | |
| | helicase, BS, RECQ2, | | |
| | RECQL2, RECQL3 | | |
| TOP3A | DNA topoisomerase IIIα, | 7156 | 27 |
| | TopIIIα, TOP3, ZGRF7 | | |
| RMI2 | RecQ-mediated genome | 116028 | 28 |
| | instability 2, BLAP18, | | |
| | C16orf75 | | |

Reducing (Inhibiting) Expression and/or Function of an Endogenous Gene

In some case a subject method includes introduction of an agent that inhibits expression and/or function of an endogenous gene. In some case a subject method includes introduction of an agent that inhibits expression of an endogenous gene. In some case a subject method includes introduction of an agent that inhibits function of an endogenous gene. In some cases a subject method includes introduction of an agent that reduces/inhibits expression of an endogenous gene and/or reduces/inhibits function of a gene product (e.g., a protein) encoded by the endogenous gene (e.g., a small molecule/drug, and antibody, and the like).

Reducing (inhibiting) expression and/or function of a gene herein refers to reducing protein production (the gene's expression) from the endogenous locus and/or inhibiting the function of the protein that is produced from the endogenous locus (e.g., via genetic mutation resulting in partial or total loss of function allele(s), via small molecule drug, antibody, and the like). Reducing function of an endogenous gene can be considered to encompass inhibiting/reducing expression of the gene (e.g., by reducing the total amount of protein produced) as well inhibiting/reducing function of a gene product (e.g., protein) encoded/produced by the endogenous gene (e.g., using a small molecule drug, antibody, etc.)—either way, the overall level of function provided by the endogenous locus is reduced/inhibited/blocked.

As would be readily understood by one of ordinary skill in the art, one can reduce expression (protein production) of an endogenous gene at the DNA, RNA, or protein level. For example, expression can be reduced by reducing the total amount of wild type protein made by the endogenous locus, and this can be accomplished either by changing the nature of the protein produced (e.g., via gene mutation to generate a loss of function allele such as a null allele or an allele that encodes a protein reduced function) or by reducing the overall levels of protein produced without changing the nature of the protein itself.

Reducing (inhibiting) expression and/or function of an endogenous gene can be accomplished using any convenient method and one of ordinary skill in the art will be aware of multiple suitable methods. For example, in order to reduce/inhibit expression, one can reduce protein levels post-translationally; one can block production of protein by blocking/reducing translation of mRNA (e.g., using an RNAi agent such as an shRNA or siRNA that targets the mRNA of an endogenous gene); one can reduce mRNA levels post-transcriptionally (e.g., using an RNAi agent such as an shRNA or siRNA that targets the mRNA of an endogenous gene); one can reduce mRNA levels by blocking transcription (e.g., using gene editing tools to either alter a promoter and/or enhancer sequence or to modulate transcription, or by using modified gene editing tools, e.g., CRISPRi, that can modify transcription without cutting the target DNA). Additionally, one can alter the nature of the protein made from an endogenous locus by inducing (e.g., using gene editing technology) a loss of function mutation, which can range from an allele with reduced wild type activity to a dead protein or no protein (e.g., catalytically inactive mutant, a frameshift allele, a gene knockout, etc). Moreover, one can reduce mRNA levels via gene editing methods that result in low net transcript levels (e.g., frameshift mutations can trigger nonsense mediated mRNA decay).

Examples of agents that inhibit expression and/or function of an endogenous gene (see above) include but are not limited to: (a) an RNAi agent such as an shRNA or siRNA that specifically targets mRNA encoded by the endogenous gene; (b) a genome editing agent (e.g., a Zinc finger nuclease, a TALEN, a CRISPR/Cas genome editing agent such as Cas9, Cpf1, CasX, CasY, and the like) that cleaves the target cell's genomic DNA at a locus encoding the endogenous gene (e.g., FANCM)—thus inducing a genome editing event (e.g., null allele, partial loss of function allele) at the locus of the endogenous gene; (c) a modified genome editing agent such as a nuclease dead zinc finger, TALE, or CRISPR/Cas nuclease fused to a transcriptional repressor protein that modulates (e.g. reduces) transcription at the locus encoding the endogenous gene (e.g., FANCM) (see, e.g., Qi et al., Cell. 2013 Feb. 28; 152(5):1173-83′; Gilbert et al, Cell. 2014 Oct. 23; 159(3):647-61; Larson et al., Nat Protoc. 2013 November; 8(11):2180-96); and (d) a small molecule/drug that directly blocks/reduces/inhibits the function of the protein produced by the endogenous locus.

When the agent is a CRISPR/Cas editing agent, the agent can include both the protein and guide RNA component. The guide nucleic acid (e.g., guide RNA) can be introduced into the cell as an RNA or as a DNA encoding the RNA (e.g., encoded by a DNA vector—on a plasmid, virus, and the like). The CRISPR/Cas protein can be introduced into the cell as a protein or as a nucleic acid (mRNA or DNA) encoding the protein. For additional information related to programmable gene editing agents and their guide nucleic acids (e.g., CRISPR/Cas RNa-guided proteins such as Cas9, CasX, CasY, and Cpf1, Zinc finger proteins such as Zinc finger nucleases, TALE proteins such as TALENs, CRISPR/Cas guide RNAs, and the like) refer to, for example, Dreier, et al., (2001) J Biol Chem 276:29466-78; Dreier, et al., (2000) J Mol Biol 303:489-502; Liu, et al., (2002) J Biol Chem 277:3850-6); Dreier, et al., (2005) J Biol Chem 280:35588-97; Jamieson, et al., (2003) Nature Rev Drug Discov 2:361-8; Durai, et al., (2005) Nucleic Acids Res 33:5978-90; Segal, (2002) Methods 26:76-83; Porteus and Carroll, (2005) Nat Biotechnol 23:967-73; Pabo, et al., (2001) Ann Rev Biochem 70:313-40; Wolfe, et al., (2000) Ann Rev Biophys Biomol Struct 29:183-212; Segal and Barbas, (2001) Curr Opin Biotechnol 12:632-7; Segal, et al., (2003) Biochemistry 42:2137-48; Beerli and Barbas, (2002) Nat Biotechnol 20:135-41; Carroll, et al., (2006) Nature Protocols 1:1329; Ordiz, et al., (2002) Proc Natl Acad Sci USA 99:13290-5; Guan, et al., (2002) Proc Natl Acad Sci USA 99:13296-301; Sanjana et al., Nature Protocols, 7:171-192 (2012); Zetsche et al, Cell. 2015 Oct. 22; 163(3):759-71; Makarova et al, Nat Rev Microbiol. 2015 November; 13(11):722-36; Shmakov et al., Mol Cell. 2015 Nov. 5; 60(3):385-97; Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September;31(9):839-43; Qi et al, Cell. 2013 Feb. 28; 152(5):1173-83; Wang et al., Cell. 2013 May 9; 153(4):910-8; Auer et. al., Genome Res. 2013 Oct. 31; Chen et. al., Nucleic Acids Res. 2013 Nov. 1; 41(20):e19; Cheng et. al., Cell Res. 2013 October; 23(10):1163-71; Cho et. al., Genetics. 2013 November; 195(3):1177-80; DiCarlo et al., Nucleic Acids Res. 2013 April; 41(7):4336-43; Dickinson et. al., Nat Methods. 2013 October; 10(10):1028-34; Ebina et. al., Sci Rep. 2013; 3:2510; Fujii et. al, Nucleic Acids Res. 2013 Nov. 1; 41(20):e187; Hu et. al., Cell Res. 2013 November; 23(11):1322-5; Jiang et. al., Nucleic Acids Res. 2013 Nov. 1; 41(20):e188; Larson et. al., Nat Protoc. 2013 November; 8(11):2180-96; Mali et. at., Nat Methods. 2013 October; 10(10):957-63; Nakayama et. al., Genesis. 2013 December; 51(12):835-43; Ran et. al., Nat Protoc. 2013 November; 8(11):2281-308; Ran et. al., Cell. 2013 Sep. 12; 154(6):1380-9; Upadhyay et. al., G3 (Bethesda). 2013 Dec. 9; 3(12):2233-8; Walsh et. al., Proc Natl Acad Sci USA. 2013 Sep. 24; 110(39):15514-5; Xie et. al., Mol Plant. 2013 Oct. 9; Yang et. al., Cell. 2013 Sep. 12; 154(6):1370-9; Briner et al., Mol Cell. 2014 Oct. 23; 56(2):333-9; Burstein et al., Nature. 2016 Dec. 22—Epub ahead of print; Gao et al., Nat Biotechnol. 2016 July 34(7):768-73; as well as international patent application publication Nos. WO2002099084; WO00/42219; WO02/42459; WO2003062455; WO03/080809; WO05/014791; WO05/084190; WO08/021207; WO09/042186; WO09/054985; and WO10/065123; U.S. patent application publication Nos. 20030059767, 20030108880, 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; 20140377868; 20150166983; and 20160208243; and U.S. Pat. Nos. 6,140,466; 6,511,808; 6,453,242 8,685,737; 8,906,616; 8,895,308; 8,889,418; 8,889,356; 8,871,445; 8,865,406; 8,795,965; 8,771,945; and 8,697,359; all of which are hereby incorporated by reference in their entirety.

### Donor DNA

The term “donor DNA” is used herein to mean a DNA molecule with a nucleic acid sequence (“donor sequence”) to be inserted (via homologous recombination) into a target site of a target DNA molecule (e.g., the genomic DNA of a target cell). In some cases the donor sequence is inserted at or near a cleavage site induced by a site specific genome editing protein (e.g., a programmable genome editing protein such as a CRISPR/Cas protein). The donor DNA will contain sufficient homology to a genomic sequence at the target site, e.g. 70% or more (such as 80% or more, 85% or more, 90% or more, 95% or more, or 98% or more) homology with the nucleotide sequence of the target site (e.g., homology with sequences flanking the cleavage site, e.g. within about 50 bases or less of the cleavage site, e.g. within about 30 bases, within about 15 bases, within about 10 bases, within about 5 bases, or immediately flanking the cleavage site) to support homologous recombination between the donor DNA and the target DNA (e.g., genomic DNA) to which it bears homology.

In some cases the donor DNA is 25 or more nucleotides (nt) (base pairs if the donor DNA is double stranded) (e.g., 50 or more, 100 or more, 200 or more, 500 or more, 1000 or more, or 2500 or more nt) in length. In some cases the donor DNA has 25 or more nucleotides (nt) (base pairs if the donor DNA is double stranded) (e.g., 50 or more, 100 or more, 200 or more, 500 or more, 1000 or more, or 2500 or more nt) of sequence homology between the donor DNA and a target sequence (e.g., a target genomic sequence). In some cases the donor DNA is 25 nt to 5 kb long (base pair if it is double stranded) (e.g., 25 nt to 3 kb, 25 nt to 2 kb, 25 nt to 1 kb, 25 nt to 800 nt, 25 nt to 600 nt, 25 nt to 500 nt, 25 nt to 400 nt, 50 nt to 5 kb, 50 nt to 3 kb, 50 nt to 2 kb, 50 nt to 1 kb, 50 nt to 800 nt, 50 nt to 600 nt, 50 nt to 500 nt, 50 nt to 400 nt, 100 nt to 5 kb, 100 nt to 3 kb, 100 nt to 2 kb, 100 nt to 1 kb, 100 nt to 800 nt, 100 nt to 600 nt, 100 nt to 500 nt, 100 nt to 400 nt, 200 nt to 5 kb, 200 nt to 3 kb, 200 nt to 2 kb, 200 nt to 1 kb, 200 nt to 800 nt, 200 nt to 600 nt, 200 nt to 500 nt, or 200 nt to 400 nt long).

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homologous recombination. In some embodiments, the donor sequence comprises a non-homologous sequence flanked by two regions of homology, such that homologous recombination between the target DNA region and the two flanking sequences results in insertion of the non-homologous sequence at the target region. Donor DNAs may also include a vector backbone containing sequences that are not homologous to the target DNA region of interest and that are not intended for insertion into the DNA region of interest. Generally, the homologous region(s) of a donor sequence can have at least 50% (e.g., at least 60%, 70%, 80%, 90%, 95%, 98%, 99%, or 99.5%) sequence identity to a genomic sequence with which recombination is desired.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein).

The donor sequence may be provided to the cell as single-stranded (ss) or double-stranded (ds) DNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues can be added to the 3′ terminus of a linear molecule and/or self-complementary oligonucleotides can be ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by viruses (e.g., adenovirus, AAV). Any convenient AAV serotype can be used for methods of nucleic acid delivery. For example, in some cases, the AAV used is serotype AAV1, AAV2, AAV3, AAV3B, AAV4, AAV5, AAV6, AAV7, AAV8, AAV9, AAV10, or AAV11 (see, e.g, Barzel et al., Nature. 2015 Jan. 15; 517(7534):360-4). In some cases, the serotype used is AAV2.

### Inhibition of Centriole Assembly

The discovery by the inventors that reduced expression/function of genes/proteins such as SASS6, SPC24, and CENPM leads to enhanced homologous recombination indicated that inhibition of centriole/centrosome assembly can also enhance homologous recombination. Thus, in some embodiments, a subject method (e.g., a method of inducing homologous recombination in a target cell, a method of editing a genome, a method of enhancing homologous recombination in a target cell, and the like) includes introducing into a target cell: (a) a donor DNA (in some cases introduced into the cell by a virus such as AAV); and (b) an agent that inhibits centriole/centrosome assembly (e.g., an inhibitor of Polo-like kinase 4 (Plk4), centrionone, centrinone B, CFI-400945, R1530, and the like).

### Cleavage of Target DNA

In any of the above methods (e.g., where a agent that inhibits centriole assembly is introduced, where an agent that inhibits expression and/or function of an endogenous gene is introduced, and the like), the method can be performed by cleaving (e.g., single strand nick or double strand break) the target site (the site with homology to the donor DNA), e.g., by introducing a programmable DNA editing endonuclease (with the appropriate guide nucleic acid in the case of a CRISPR/Cas endonuclease). Thus, in some cases a subject method includes cleavage of the target cell's genomic DNA with a gene editing endonuclease (e.g., a CRISPR/Cas programmable endonuclease, a zinc finger nuclease (ZFN), a TALE nuclease (TALEN), and the like) at a locus with homology to the donor DNA, e.g., prior to incorporation of the sequence of the donor DNA. In some cases where the genome is cleaved prior to HR, the subject methods can reduce the chances of undesired non-homologous end-joining (NHEJ), which is a DNA repair pathway the cell can use instead of HR.

The act of cleaving a target cell's genomic DNA at a locus with homology to the donor DNA can be performed by introducing the appropriate components into the target cell. For example, in some cases a subject method includes introducing into the target cell: (a) a donor DNA; (b) an agent that inhibits expression and/or function of an endogenous gene (e.g., selected from the group consisting of: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and COPZ1; selected from the group consisting of: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2; and the like); and (c) a genome editing agent (e.g., ZFN, TALEN, CRISPR/Cas programmable nuclease and an appropriate guide nucleic acid, and the like). in some cases a subject method includes introducing into the target cell: (a) a donor DNA; (b) an agent or agents that inhibit expression and/or function of two or more endogenous genes (e.g., where one is FANCM and the other is encodes a member of the BTR complex; where one is FANCM and the other is RMI1; where one is FANCM and the other is BLM; and the like); and (c) a genome editing agent (e.g., ZFN, TALEN, CRISPR/Cas programmable nuclease and an appropriate guide nucleic acid, and the like). As noted elsewhere in this disclosure the genome editing agents can be introduced as DNA, RNA, and/or protein as desired.

In any of the subject methods (e.g., where a agent that inhibits centriole assembly is introduced, where an agent that inhibits expression and/or function of an endogenous gene is introduced, and the like), the method can also be performed without cleaving the target site at the site of the target DNA having homology with the donor DNA. This can be accomplished, e.g., using recombinant adeno-associated virus (rAAV)-mediated gene targeting without nucleases—see Barzel et al., Nature. 2015 Jan. 15; 517(7534):360-4).

Thus, in some cases a subject method does not include a step of cleaving the target cell's genomic DNA prior to incorporation of the sequence of the donor DNA (e.g., the target cell's genome is not cleaved with a gene editing endonuclease at a locus with homology to the donor DNA prior to incorporation of the sequence of the donor DNA).

### Target Cells

The cells of interest (i.e., “target cells”) are typically vertebrate cells (e.g., mammalian cells). Mammalian cells refers to cells of any animal classified as a mammal, including humans, domestic and farm animals, and zoo, laboratory, sports, or pet animals, such as dogs, horses, cats, cows, mice, rats, rabbits, primates, non-human primates etc. In some embodiments, the target cell is a human cell. In some cases the target cell is in vivo (e.g., the methods can be used as therapeutic gene editing methods). In some cases, the target is a cell removed from an individual (e.g., a “primary” cell) (e.g., a cell ex vivo). In some cases, the target cell is a tissue culture cell (e.g., from an established cell line) (e.g., a cell in vitro).

Exemplary target cells include, but are not limited to, liver cells, pancreatic cells (e.g., islet cells: alpha cells, beta cells, delta cells, gamma cells, and/or epsilon cells), skeletal muscle cells, heart muscle cells, kidney cells, fibroblasts, retinal cells, synovial joint cells, lung cells, T cells, neurons, glial cells, stem cells, blood cells, leukocytes, hematopoietic stem cells, hematopoietic progenitor cells, myeloid cells, immune cells, neural progenitor cells, endothelial cells, and cancer cells. Exemplary stem cell target cells include, but are not limited to, hematopoietic stem cells, neural stem cells, neural crest stem cells, embryonic stem cells, induced pluripotent stem cells (iPS cells), mesenchymal stem cells, mesodermal stem cells, liver stem cells, pancreatic stem cells, muscle stem cells, and retinal stem cells.

Introduction into Cells/Administration

In some embodiments, a donor DNA and/or an agent is introduced into a cell. In some cases, the cell is in vivo. For example, in some cases, ‘introducing into a target cell’ (e.g., introducing a donor DNA and/or an agent, e.g., an agent that inhibits expression and/or function of an endogenous gene such as an RNAi agent, an agent that inhibits centriole assembly, and the like into a target cell) is achieved by administering the donor DNA and/or agent to an individual. In some cases, introducing into a cell includes administering to an individual. The donor DNA and/or agent can be administered in a series of more than one administration. For example, they can be delivered simultaneously, as part of the same mixture/composition, one after the other, etc.

In some cases, introducing a donor DNA and/or an agent into a target cell includes inducing expression of the agent. For example, in some cases the agent is an RNAi agent (e.g., shRNA), which can be encoded in a DNA molecule, and the DNA molecule encoding the RNAi agent, and expression of the RNAi agent from the DNA molecule can be induced (e.g., in cases where a nucleotide sequence encoding the agent is operably linked to an inducible promoter). Thus in some cases, a subject method includes inducing expression of a subject agent (e.g., an agent that inhibits expression and/or function of an endogenous gene). In some cases such a step is followed or preceded by a step of introducing the donor DNA.

Compositions for administration (e.g., a donor DNA and/or a subject agent such as an agent that inhibits expression and/or function of an endogenous gene or an agent that inhibits centriole assembly) can be administered systemically or locally (e.g., directly to the tissue in which HR is desired). In some cases, compositions for administration are administered by parenteral, topical, intravenous, intratumoral, oral, subcutaneous, intraarterial, intracranial, intraperitoneal, intranasal or intramuscular means. A typical route of administration is intravenous or intratumoral, although other routes can be equally effective. For example, the cells and/or compositions may be introduced to the subject (i.e., administered to the individual) via any of the following routes: parenteral, subcutaneous (s.c.), intravenous (i.v.), intracranial (i.c.), intraspinal, intraocular, intradermal (i.d.), intramuscular (i.m.), intralymphatic intratumoral, or into spinal fluid. The cells and/or compositions may be introduced by injection (e.g., systemic injection, direct local injection, local injection into or near a tumor and/or a site of tumor resection, etc.), catheter, or the like. Examples of methods for local delivery (e.g., delivery to a tumor and/or cancer site) include, e.g., by bolus injection, e.g. by a syringe, e.g. into a joint, tumor, or organ, or near a joint, tumor, or organ; e.g., by continuous infusion, e.g. by cannulation.

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this disclosure can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

An “effective dose” is an amount sufficient to effect desired biological result (e.g., facilitating HR, increasing the frequency of HR, achieving genome editing, and the like). A “therapeutically effective dose” or “therapeutic dose” is an amount sufficient to effect desired clinical results (i.e., achieve therapeutic efficacy, e.g., reduce/halt/slow a cancer, reduce/halt/slow an infectious disease, and the like). A therapeutically effective dose can be administered in one or more administrations. For purposes of this disclosure, a therapeutically effective dose of a subject compositions is an amount that is sufficient, when administered to (e.g., introduced into a cell of) the individual, to palliate, ameliorate, stabilize, reverse, prevent, slow or delay the progression of the disease state (e.g., tumor size, tumor growth, tumor presence, cancer presence, presence of infectious disease, presence of pathogen, etc.) by, for example, facilitating HR in target cells.

Therapeutic entities of the present disclosure can be administered on multiple occasions. Intervals between single dosages can be daily, weekly, monthly or yearly. Intervals can also be irregular as indicated by measuring blood levels of the therapeutic entity in the patient. Alternatively, therapeutic entities of the present disclosure can be administered as a sustained release formulation, in which case less frequent administration is required. Dosage and frequency can vary depending on the half-life of the compositions administered to the patient.

Agents of the disclosure (donor DNA and/or an agent that inhibits expression and/or function of an endogenous gene, e.g., an RNAi agent) can be introduced into a cell using any convenient method and many such methods will be readily available to one of ordinary skill in the art. For example, methods of introducing nucleic acids include but are not limited to: electroporation, transfection, direct injection (e.g., microinjection), lipid-based delivery, nanoparticle delivery, viral delivery, and the like. When using viral delivery (e.g., to deliver a donor DNA and/or an RNAi agent and/or a gene editing agent such as a nucleic acid encoding a guide nucleic acid and/or encoding an endonuclease such as a CRISPR/Cas nuclease, ZFN, TALEN, etc), any convenient viral vector can be used (e.g., lentivirus, retrovirus, adenovirus, adeno-associated virus (AAV), vaccinia virus; poliovirus; a retroviral vector, Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, mammary tumor virus, and the like). When using AAV, any convenient AAV serotype can be used, e.g., AAV1, AAV2, AAV3, AAV3B, AAV4, AAV5, AAV6, AAV7, AAV8, AAV9, AAV10, or AAV11. In some cases, the serotype used is AAV2.

### Genetically Modified Cells

The present disclosure provides genetically modified cells with enhanced HR. Such cells can have a reduced wild type protein level from the endogenous locus (e.g., due to an altered nucleotide sequence at an endogenous genomic locus, due to an RNAi agent that specifically targets expression of an HR gene, etc.) of one or more genes selected from: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and COPZ1. In some cases, a genetically modified cell with enhanced HR has a reduced wild type protein level from the endogenous locus of one or more genes selected from: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and COPZ1. In some cases, a genetically modified cell with enhanced HR has a reduced wild type protein level from the endogenous locus of one or more genes selected from: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2. In some cases, a genetically modified cell with enhanced HR has a reduced wild type protein level from the endogenous locus of one or more genes selected from: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2. In some cases, a genetically modified cell with enhanced HR has a reduced wild type protein level from the endogenous locus of one or more genes selected from: FANCM, DDX28, PCBP1, SFRS1/SRSF1, CDK19, DNAJA3/TID1, CDC16, and CDT1. In some cases, a genetically modified cell with enhanced HR has a reduced wild type protein level from the endogenous locus of one or more genes selected from: SASS6, SPC24, and CENPM. In some cases, a genetically modified cell with enhanced HR has a reduced wild type protein level from the endogenous locus of one or more genes selected from: FANCM, RMI1, BLM, TOP3A, and RMI2. In some cases, a genetically modified cell with enhanced HR has a reduced wild type protein level from the endogenous locus of one or more genes selected from: FANCM and RMI1. In some cases, a genetically modified cell with enhanced HR has a reduced wild type protein level from the FANCM endogenous locus.

In some cases, a genetically modified cell with enhanced HR has a reduced wild type protein level from the endogenous locus (e.g., due to an altered nucleotide sequence at an endogenous genomic locus, due to an RNAi agent or nucleic acid encoding said RNAi agent where the RNAi agent specifically targets expression of the endogenous gene, and the like) of two or more genes, where one gene is FANCM and another encodes a member of the BRR complex (e.g., RMI1 or BLM). As such, in some cases one gene is FANCM and another is RMI1. In some cases one gene is FANCM and another is BLM.

Aspects of the disclosure include a genetically modified mammalian cell with enhanced HR that includes one or more of: (a) an altered nucleotide sequence at an endogenous genomic locus of an endogenous gene (same options as in the paragraph above, e.g., in some cases the endogenous gene is selected from: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and COPZ1; FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2; and the like) compared to a corresponding endogenous genomic locus of a corresponding wild type cell; and (b) an RNAi agent, or nucleic acid encoding said RNAi agent, wherein the RNAi agent specifically targets expression of the endogenous gene, where (a) and (b), independently or combined, cause a reduced wild type protein level from the endogenous locus in the genetically modified mammalian cell relative to the protein level in the absence of (a) and (b). In some cases, the genetically modified mammalian cell includes a deletion of exon sequence at the endogenous genomic locus. In some cases, the genetically modified mammalian cell includes the nucleic acid encoding the RNAi agent. In some cases, the nucleic acid encoding the RNAi agent is integrated into the genome of the genetically modified cell. In some cases it is not integrated.

Genetically modified cells (including isolated genetically modified cells) can a include foreign nucleic acid such as a foreign DNA that includes a nucleotide sequence encoding an RNAi agent and/or can have an altered sequence in the genome (e.g., a loss of function allele, a knock-out/null allele, etc.) at one or more endogenous loci.

In some embodiments, a subject genetically cell includes a foreign nucleic acid such as an RNAi agent (e.g., shRNA, siRNA, microRNA) or a DNA encoding an RNAi agent (e.g., episomally, integrated into the genome) where the RNAi agent specifically targets one or more of the cell's endogenous gene/proteins—in some cases selected from: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and COPZ1; in some cases selected from: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2; in some cases selected from: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and COPZ1; in some cases selected from: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2; in some cases selected from: FANCM, RMI1, BLM, TOP3A, and RMI2; in some cases selected from: FANCM and RMI1; in some cases selected from: FANCM, DDX28, PCBP1, SFRS1/SRSF1, CDK19, DNAJA3/TID1, CDC16, and CDT1; and in some cases selected from: SASS6, SPC24, and CENPM. In some cases, a subject genetically cell includes an RNAi agent (e.g., shRNA, siRNA, microRNA) or a nucleic acid encoding an RNAi agent (e.g., episomally, integrated into the genome) where the RNAi agent specifically targets FANCM. In some cases the foreign nucleic acid (e.g., DNA encoding an RNAi agent) is incorporated into the cell's genome. In some cases, the foreign nucleic acid (e.g., DNA encoding an RNAi agent) is maintained episomally. In some cases, the foreign nucleic acid (e.g., RNAi agent) is transiently present in the cell.

Any cell type can be a genetically modified cell. Cells of interest include vertebrate cells (e.g., mammalian cells). Mammalian cells refers to cells of any animal classified as a mammal, including humans, domestic and farm animals, and zoo, laboratory, sports, or pet animals, such as dogs, horses, cats, cows, mice, rats, rabbits, primates, non-human primates etc. In some embodiments, the cell is a human cell. In some cases, the cell ex vivo). In some cases, the target cell is a tissue culture cell (e.g., an in vitro cell), in some cases the cell is a primary cell (e.g., ex vivo). Cells of interest include, but are not limited to, liver cells, pancreatic cells (e.g., islet cells: alpha cells, beta cells, delta cells, gamma cells, and/or epsilon cells), skeletal muscle cells, heart muscle cells, kidney cells, fibroblasts, retinal cells, synovial joint cells, lung cells, T cells, neurons, glial cells, stem cells, blood cells, leukocytes, hematopoietic stem cells, hematopoietic progenitor cells, myeloid cells, immune cells, neural progenitor cells, endothelial cells, and cancer cells. Exemplary stem cell target cells include, but are not limited to, hematopoietic stem cells, neural stem cells, neural crest stem cells, embryonic stem cells, induced pluripotent stem cells (iPS cells), mesenchymal stem cells, mesodermal stem cells, liver stem cells, pancreatic stem cells, muscle stem cells, and retinal stem cells.

In some embodiments, a subject genetically modified cell is a vertebrate cell or is derived from a vertebrate cell. In some embodiments, a subject genetically modified cell is a mammalian cell or is derived from a mammalian cell. In some embodiments, a subject genetically modified cell is a rodent cell (e.g., a mouse cell, a rat cell, and the like) or is derived from a rodent cell. In some embodiments, a subject genetically modified cell is a human cell or is derived from a human cell. In some embodiments, a subject genetically modified cell is a genetically modified stem cell or progenitor cell. Suitable cells include, e.g., stem cells (adult stem cells, embryonic stem cells, iPS cells, etc.) and progenitor cells (e.g., cardiac progenitor cells, neural progenitor cells, etc.). Suitable cells include mammalian stem cells and progenitor cells, including, e.g., rodent stem cells, rodent progenitor cells, human stem cells, human progenitor cells, etc. Suitable cells include in vitro cells, e.g., isolated cells.

The present disclosure further provides progeny of a subject genetically modified cell, where the progeny can comprise the same exogenous nucleic acid and/or genomic alteration as the subject genetically modified cell from which it was derived. The present disclosure further provides a composition comprising a subject genetically modified cell.

### Genetically Modified Non-Human Mammals

Provided are non-human genetically modified organisms (e.g., mammal, rodent, mouse, rat, pig, horse, sheep, cow, ungulate, non-human primate) that (1) includes an exogenous nucleic acid comprising a nucleotide sequence encoding an agent that inhibits expression and/or function of an endogenous gene selected from FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and COPZ1 (e.g., RNAi agent); and/or (2) has been genetically modified at the endogenous locus encoding an endogenous gene (e.g., FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, and/or COPZ1) to decrease wild type expression of the gene encoded at the locus. Provided are non-human genetically modified organisms (e.g., mammal, rodent, mouse, rat, pig, horse, sheep, cow, ungulate, non-human primate) that (1) includes an exogenous nucleic acid comprising a nucleotide sequence encoding an agent that inhibits expression and/or function of an endogenous gene selected from FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2 (e.g., RNAi agent); and/or (2) has been genetically modified at the endogenous locus encoding an endogenous gene (e.g., FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and/or RMI2) to decrease wild type expression of the gene encoded at the locus. Examples of suitable non-human genetically modified organisms include but are not limited to: mammals, rodents, mice, rats, pigs, horses, sheep, cows, ungulates, and non-human primates.

If such a non-human genetically modified organism includes an exogenous nucleic acid comprising a nucleotide sequence encoding an agent (e.g., RNAi agent) that inhibits expression and/or function of one or more of the endogenous genes listed above, the exogenous nucleic acid can be extrachromosomal (e.g., episomal) or can be integrated into the genome. In some cases the exogenous nucleic acid is operably linked to a functioning promoter.

In some embodiments, a subject genetically modified non-human cell (e.g., a cell that has been genetically modified with an exogenous nucleic acid, a cell that has an altered genomic sequence at an endogenous locus such as one that encodes FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, or COPZ1; a cell that has an altered genomic sequence at an endogenous locus such as one that encodes FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, or RMI2) can be used to generate a subject genetically modified non-human organism (e.g., a rodent, a rat, a mouse, a non-human primate, a mammal, etc.). For example, if the genetically modified cell is a pluripotent stem cell (i.e., PSC) or a germ cell (e.g., a spermatogonium, a sperm, an oogonium, an oocyte, etc.), an entire genetically modified organism can be derived from the genetically modified cell. In some embodiments, the genetically modified cell is a pluripotent stem cell (e.g., ESC, iPSC, pluripotent plant stem cell, etc.) or a germ cell (e.g., a spermatogonium, a sperm, an oogonium, an oocyte, etc.) either in vivo or in vitro that can give rise to a genetically modified organism. In some embodiments the genetically modified cell is a vertebrate pluripotent stem cell (PSC) (e.g., ESC, iPSC, etc.) and is used to generate a genetically modified organism (e.g. by injecting a PSC into a blastocyst to produce a chimeric/mosaic animal, which could then be mated to generate non-chimeric/non-mosaic genetically modified organisms; grafting in the case of plants; etc.).

### Kits

Also within the scope of the disclosure are kits comprising the compositions described herein. Non-limiting examples of components that can be included in a kit include but are not limited to (and can be included in any convenient combination): (a) a donor DNA; (b) a CRISPR/Cas guide nucleic acid (e.g., guide RNA) that targets the endogenous genomic locus encoding FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, or COPZ1; (c) an RNAi agent, or nucleic acid encoding an RNAi agent, where the RNAi agent specifically targets an mRNA encoding FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, or COPZ1; (d) a CRISPR/Cas guide nucleic acid (e.g., guide RNA) that targets a genomic sequence with which the donor DNA has homology (which can be used for e.g., cleaving DNA at the site at which homologous recombination is desired); (e) an agent that inhibits centriole assembly (e.g., an inhibitor of Polo-like kinase 4 (Plk4), centrionone, centrinone B, CFI-400945, and/or R1530); (f) a eukaryotic cell (e.g., mammalian cell); (g) a nucleic acid sequence encoding a nuclease (e.g., selected from: a CRISPR/Cas programmable endonuclease such as Cas9, Cpf1, CasX, and CasY; a ZFN, a TALEN, and the like); (h) a viral vector (e.g., an AAV vector); (i) an agent such as a small molecule (drug) that blocks/inhibits/reduces the function of a protein produced from one of the following endogenous loci: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, or COPZ1; (j) a CRISPR/Cas guide nucleic acid (e.g., guide RNA) that targets the endogenous genomic locus encoding FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, or RMI2; (k) an RNAi agent, or nucleic acid encoding an RNAi agent, where the RNAi agent specifically targets an mRNA encoding FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, or RMI2; (I) an agent such as a small molecule (drug) that blocks/inhibits/reduces the function of a protein produced from one of the following endogenous loci: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, or RMI2; and (m) an RNAi agent, or nucleic acid encoding an RNAi agent, where the RNAi agent specifically targets an mRNA encoding FANCM and one or more mRNAs that encode one or more members of the BTR complex (e.g., RMI1, BLM). Kits typically include a label indicating the intended use of the contents of the kit, and can include instructions for use. The term label includes any writing, or recorded material supplied on or with the kit, or which otherwise accompanies the kit.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure, e.g., numbered 1-86, are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of inducing homologous recombination in a target cell, the method comprising introducing into the target cell: (a) a donor DNA, and (b) an agent that inhibits expression and/or function of an endogenous gene, or inhibits function of a gene product encoded by the endogenous gene, wherein the endogenous gene is selected from the group consisting of: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2, whereby a sequence of the donor DNA is incorporated into the target cell's genomic DNA.
2. The method of 1, wherein the endogenous gene is selected from the group consisting of: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2.
3. The method of 1, wherein the endogenous gene is selected from the group consisting of: FANCM, DDX28, PCBP1, SFRS1/SRSF1, CDK19, DNAJA3/TID1, CDC16, and CDT1.
4. The method of 1, wherein the endogenous gene is selected from the group consisting of: SASS6, SPC24, and CENPM.
5. The method of 1, wherein the endogenous gene is selected from the group consisting of: FANCM, RMI1, BLM, TOP3A, and RMI2.
6. The method of 1, wherein the endogenous gene is FANCM or RMI1.
7. The method of 1, wherein the endogenous gene is FANCM.
8. The method of any one of 1-7, wherein said agent is an RNAi agent that specifically targets mRNA encoded by the endogenous gene.
9. The method of any one of 1-7, wherein said agent is a genome editing agent that cleaves the target cell's genomic DNA at a locus encoding the endogenous gene.
10. The method of 9, wherein said genome editing agent is a CRISPR/Cas genome editing agent.
11. The method of 9, wherein said genome editing agent is a Zinc finger or TALE genome editing agent.
12. The method of any one of 1-11, wherein the target cell is a eukaryotic cell.
13. The method of any one of 1-12, wherein the target cell is a mammalian cell.
14. The method of any one of 1-13, wherein the method comprises cleavage of the target cell's genomic DNA with a gene editing endonuclease at a locus with homology to the donor DNA, prior to incorporation of the sequence of the donor DNA.
15. The method of 14, wherein the gene editing endonuclease is a CRISPR/Cas programmable endonuclease.
16. The method of 15, wherein the CRISPR/Cas programmable endonuclease is selected from: Cas9, Cpf1, CasX, and CasY.
17. The method of 14, wherein the gene editing endonuclease is a Zinc Finger Nuclease (ZFN) or a TALEN.
18. The method of any one of 1-13, wherein the target cell's genomic DNA is not cleaved with a gene editing endonuclease at a locus with homology to the donor DNA prior to said incorporation of the sequence of the donor DNA.
19. The method of any one of 1-18, wherein the donor DNA is introduced into the cell by a virus.
20. The method of 19, wherein the virus is AAV.
21. A kit comprising (a) a donor DNA, and (b) an agent that inhibits expression and/or function of an endogenous gene, or inhibits function of a gene product encoded by the endogenous gene, wherein the endogenous gene is selected from the group consisting of: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2.
22. The kit of 21, wherein the endogenous gene is selected from the group consisting of: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2.
23. The kit of 21, wherein the endogenous gene is selected from the group consisting of: FANCM, DDX28, PCBP1, SFRS1/SRSF1, CDK19, DNAJA3/TID1, CDC16, and CDT1.
24. The kit of 21, wherein the endogenous gene is selected from the group consisting of: SASS6, SPC24, and CENPM.
25. The kit of 21, wherein the endogenous gene is selected from the group consisting of: FANCM, RMI1, BLM, TOP3A, and RMI2.
26. The kit of 21, wherein the endogenous gene is FANCM or RMI1.
27. The kit of 21, wherein the endogenous gene is FANCM.
28. The kit of any one of 21-27, wherein said agent is an RNAi agent that specifically targets mRNA encoded by the endogenous gene.
29. The kit of any one of 21-27, wherein said agent is a genome editing agent that cleaves the target cell's genomic DNA at a locus encoding the endogenous gene.
30. The kit of 27, wherein said genome editing agent is a CRISPR/Cas genome editing agent, a zinc finger nuclease, or a TALEN.
31. The kit of any one of 21-30, further comprising a eukaryotic cell.
32. A method of inducing homologous recombination in a target cell, the method comprising introducing into the target cell: (a) a donor DNA, and (b) an agent that inhibits centriole assembly, whereby a sequence of the donor DNA is incorporated into the target cell's genomic DNA.
33. The method of 28, wherein the agent that inhibits centriole assembly is an inhibitor of Polo-like kinase 4 (Plk4).
34. The method of 28 or 29, wherein the agent that inhibits centriole assembly is selected from the group consisting of: centrionone, centrinone B, CFI-400945, and R1530.
35. The method of any one of 28-30, wherein the agent that inhibits centriole assembly is centrinone or centrinone B.
36. The method of any one of 28-31, wherein the target cell is a eukaryotic cell.
37. The method of any one of 28-32, wherein the target cell is a mammalian cell.
38. The method of any one of 28-33, wherein the method comprises cleavage of the target cell's genomic DNA with a gene editing endonuclease at a locus with homology to the donor DNA, prior to incorporation of the sequence of the donor DNA.
39. The method of 38, wherein the gene editing endonuclease is a CRISPR/Cas programmable endonuclease.
40. The method of 49, wherein the CRISPR/Cas programmable endonuclease is selected from: Cas9, Cpf1, CasX, and CasY.
41. The method of 38, wherein the gene editing endonuclease is a Zinc Finger Nuclease (ZFN) or a TALEN.
42. The method of any one of 32-37, wherein the target cell's genomic DNA is not cleaved with a gene editing endonuclease at a locus with homology to the donor DNA prior to said incorporation of the sequence of the donor DNA.
43. The method of any one of 32-42, wherein the donor DNA is introduced into the cell by a virus.
44. The method of 43, wherein the virus is AAV.
45. A kit comprising (a) a donor DNA, and (b) an agent that inhibits centriole assembly.
46. The kit of 45, wherein the agent that inhibits centriole assembly is an inhibitor of Polo-like kinase 4 (Plk4).
47. The kit of 45 or 46, wherein the agent that inhibits centriole assembly is selected from the group consisting of: centrionone, centrinone B, CFI-400945, and R1530.
48. The kit of any one of 45-47, wherein the agent that inhibits centriole assembly is centrinone or centrinone B.
49. A method of inducing homologous recombination in a mammalian target cell, the method comprising introducing a donor DNA into a mammalian target cell that has reduced expression and/or function of an endogenous gene, or has inhibited function of a gene product encoded by the endogenous gene, wherein the endogenous gene is selected from the group consisting of: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2,

wherein said mammalian target cell comprises at least one of: (i) a loss-of-function mutation in the endogenous gene, and (ii) an RNAi agent, or DNA encoding said RNAi agent, that specifically targets mRNA encoded by the endogenous gene;

whereby a sequence of the donor DNA is incorporated into the target cell's genomic DNA.

50. The method of 49, wherein the endogenous gene is selected from the group consisting of: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2.
51. The method of 49, wherein the endogenous gene is selected from the group consisting of: FANCM, DDX28, PCBP1, SFRS1/SRSF1, CDK19, DNAJA3/TID1, CDC16, and CDT1.
52. The method of 49, wherein the endogenous gene is selected from the group consisting of: SASS6, SPC24, and CENPM.
53. The kit of 49, wherein the endogenous gene is selected from the group consisting of: FANCM, RMI1, BLM, TOP3A, and RMI2.
54. The kit of 49, wherein the endogenous gene is FANCM or RMI1.
55. The method of 49, wherein the endogenous gene is FANCM.
56. The method of any one of 49-55, wherein said agent is an RNAi agent that specifically targets mRNA encoded by the endogenous gene.
57. The method of any one of 49-55, wherein said agent is a genome editing agent that cleaves the target cell's genomic DNA at a locus encoding the endogenous gene.
58. The method of 57, wherein said genome editing agent is a CRISPR/Cas genome editing agent.
59. The method of 57, wherein said genome editing agent is a Zinc finger or TALE genome editing agent.
60. The method of any one of 49-59, wherein the target cell is a eukaryotic cell.
61. The method of any one of 49-60, wherein the target cell is a mammalian cell.
62. The method of any one of 49-61, wherein the method comprises cleavage of the target cell's genomic DNA with a gene editing endonuclease at a locus with homology to the donor DNA, prior to incorporation of the sequence of the donor DNA.
63. The method of 62, wherein the gene editing endonuclease is a CRISPR/Cas programmable endonuclease.
64. The method of 63, wherein the CRISPR/Cas programmable endonuclease is selected from: Cas9, Cpf1, CasX, and CasY.
65. The method of 62, wherein the gene editing endonuclease is a Zinc Finger Nuclease (ZFN) or a TALEN.
66. The method of any one of 49-61, wherein the target cell's genomic DNA is not cleaved with a gene editing endonuclease at a locus with homology to the donor DNA prior to said incorporation of the sequence of the donor DNA.
67. The method of any one of 49-66, wherein the donor DNA is introduced into the cell by a virus.
68. The method of 67, wherein the virus is AAV.
69. A kit comprising (a) a donor DNA, and (b) a mammalian cell that has reduced expression and/or function of an endogenous gene, or has inhibited function of a gene product encoded by the endogenous gene, wherein the endogenous gene is selected from the group consisting of: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2,

wherein said mammalian target cell comprises at least one of: (i) a loss-of-function mutation in the endogenous gene, and (ii) an RNAi agent, or DNA encoding said RNAi agent, that specifically targets mRNA encoded by the endogenous gene.

70. The kit of 69, wherein the endogenous gene is selected from the group consisting of: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2.
71. The kit of 69, wherein the endogenous gene is selected from the group consisting of: FANCM, DDX28, PCBP1, SFRS1/SRSF1, CDK19, DNAJA3/TID1, CDC16, and CDT1.
72. The kit of 69, wherein the endogenous gene is selected from the group consisting of: SASS6, SPC24, and CENPM.
73. The kit of 69, wherein the endogenous gene is selected from the group consisting of: FANCM, RMI1, BLM, TOP3A, and RMI2.
74. The kit of 69, wherein the endogenous gene is FANCM or RMI1.
75. The kit of 69, wherein the endogenous gene is FANCM.
76. A kit comprising (a) a genome editing agent, and (b) an agent that inhibits expression and/or function of an endogenous gene, or inhibits function of a gene product encoded by the endogenous gene, wherein the endogenous gene is selected from the group consisting of: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2.
77. The kit of 76, wherein the endogenous gene is selected from the group consisting of: FANCM, DDX28, PCBP1, SFRS1/SRSF1, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2.
78. The kit of 76, wherein the endogenous gene is selected from the group consisting of: FANCM, DDX28, PCBP1, SFRS1/SRSF1, CDK19, DNAJA3/TID1, CDC16, and CDT1.
79. The kit of 76, wherein the endogenous gene is selected from the group consisting of: SASS6, SPC24, and CENPM.
80. The kit of 76, wherein the endogenous gene is selected from the group consisting of: FANCM, RMI1, BLM, TOP3A, and RMI2.
81. The kit of 76, wherein the endogenous gene is FANCM or RMI1.
82. The kit of 76, wherein the endogenous gene is FANCM.
83. The kit of 76, wherein said genome editing agent is a CRISPR/Cas genome editing agent.
84. The kit of 83, wherein the CRISPR/Cas genome editing agent is a CRISPR/Cas programmable endonuclease.
85. The kit of 83, wherein the CRISPR/Cas genome editing agent is a CRISPR/Cas guide RNA.
86. The kit of 76, wherein said genome editing agent is a Zinc Finger Nuclease (ZFN) or a TALEN.

The invention now being fully described, it will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

Experiments were performed to identify factors that negatively affect homologous recombination (HR) machinery. In other words, experiments were performed to identify factors for which a reduction from wild type levels of function leads to an increase in HR efficiency.

### Methods/Results

In order to identify new host factors associated with the HR process, an unbiased, genome-wide screening approach, based on insertional mutagenesis in haploid human cells (HAP1), was used. The HAP1 cells, which have been successfully used in several different genome-wide screens, contain mutations in more than 98% of all expressed genes and each gene is hit with an average of ˜30 insertions. On average, each cell contains one mutation. The rationale behind this experiment was that cells containing specific deleterious mutations in genes that repress HR, will be enriched in the screening, suggesting that the mutated genes within the enriched cells negatively regulate the HR process (and that mutation of the identified genes leads to an increased HR efficiency).

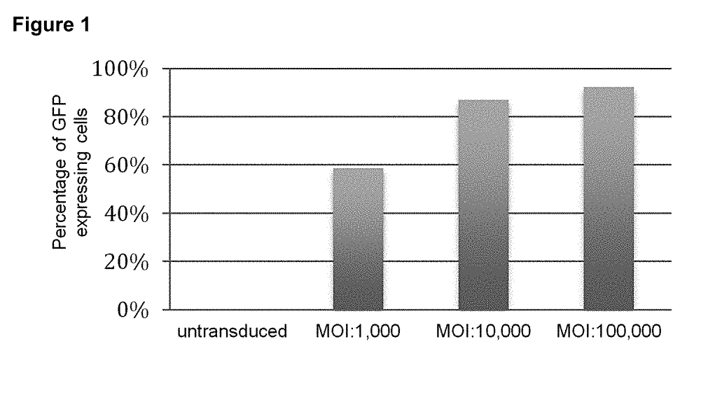

A library of mutagenized cells, carrying knockouts in virtually all non-essential genes, was infected with an adeno-associated virus (AAV) targeting construct packaged into the AAV-DJ serotype. This serotype was chosen because it transduced approximately 90% of the cells when a MOI of 10,000 was used (FIG. 1).

An AAV targeting construct was designed to target via HR the highly expressed human Glyceraldehyde-3-Phosphate Dehydrogenase (GAPDH) gene and to drive the expression of the drug resistance marker—Blasticidin (BLAS)—upon precise GAPDH locus integration (FIG. 2). This construct did not contain a promoter region, thus gene expression was dependent on the correct integration under the GAPDH locus, remarkably reducing the likelihood of false-positives.

Screening was performed in two experimental duplicates. Two sets of eleven T175 cm² flasks containing 13×10⁶ cells were transduced with the AAV-GAPDH-2A-BLAS vector using an MOI of 16,000. Forty-eight hours after AAV transduction, two drug selections were used: 20 μg/mL (more stringent) and 5 μg/mL (less stringent) of blasticidin. Cells were cultured for seven days with blasticidin, which was shown to be effective to eliminate cells without the BLAS resistance.

Cells were harvested by trypsin treatment and DNA was extracted from 30×10⁶ cells for Linear Amplification Mediated PCR (LAM-PCR) and deep-sequencing analysis (miseq—IIlumina platform). After performing bioinformatics analysis, the LRRC8 gene, which was previously found to be required for blasticidin import and therefore served as a positive control for the screening experiment, was enriched in both screenings as expected. Many mutated genes were enriched (with small p-values) in both screenings, suggesting a strong link between those genes and HR events in the cells. For further analysis, initially eight genes were selected based on their function and cellular location of their expressed proteins (Table 2).

TABLE 2
Enriched genes identified in both HAP1 cell screenings
for factors that negatively affect the HR machinery after
transduction of the AAV-GAPDH-2A-BLAS vector.

| | | Enrichment | Enrichment |
|---|---|---|---|
| | | position | position |
| Enriched | | (5 μg/mL | (20 μg/mL |
| Genes | Full name | screen) | screen) |
| CDK19 | cyclin dependent kinase 19 | 3 | 32 |
| DNAJA3 | DnaJ heat shock protein family | 4 | 54 |
| | (Hsp40) member A3 | | |
| SFRS1 | serine and arginine rich | 5 | 15 |
| (SRSF1) | splicing factor 1 | | |
| CDC16 | cell division cycle 16 | 6 | 350 |
| PCBP1 | poly(rC) binding protein 1 | 10 | 3 |
| DDX28 | DEAD-box helicase 28 | 51 | 2 |
| FANCM | Fanconi anemia | 157 | 6 |
| | complementation group M | | |
| CDT1 | chromatin licensing and DNA | 170 | 10 |
| | replication factor 1 | | |

In a second step, more genes were selected. In order to prioritize the most statistically significant hits, genes were selected which were shared in both screenings within the top 400 positions. This provided a list of 90 genes, which included those that are also listed in Table 2. Next, genes were removed from this list genes that: (i) had unknown functions, (ii) had functions which were not directly associated with HR events and/or (iii) have been hits in previous unrelated HAP1 screenings more than once (e.g., possibly false positives). After this filtering, a list of 24 genes was compiled (see Table 1 above).

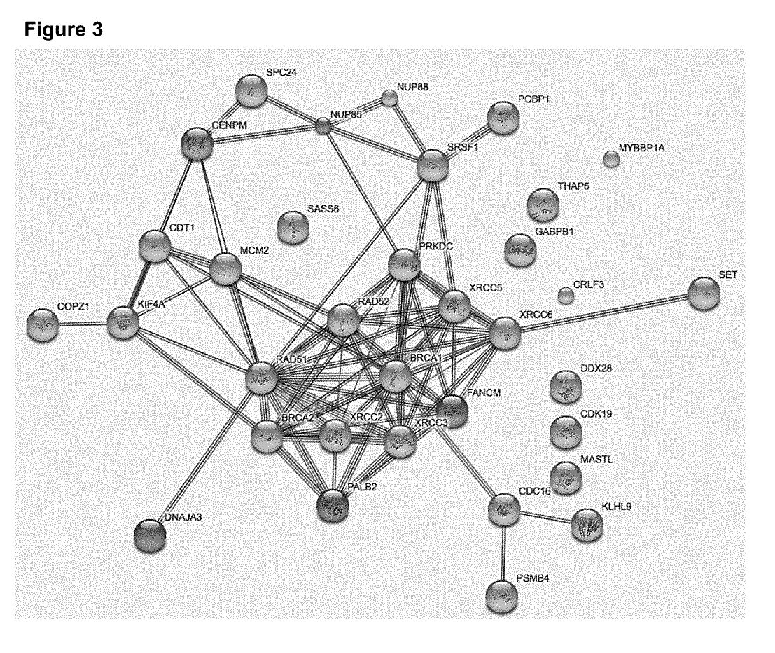

Next, the 24 selected genes from Table 1 together with genes known to be associated with both HR (PALB2, RAD52, RAD51, BRCA1 and BRCA2) and NHEJ (XRCC6 (also known as Ku70), XRCC5 (also known as Ku86) and PRKDC (also known as DNA-PK)) pathways were entered in the String software and a model of protein-protein interactions was generated (FIG. 3).

Interestingly, 16 of the 24 selected genes seemed to be directly or indirectly associated with the homologous recombination (HR) and non-homologous end joining (NHEJ) pathways, which strongly supports the relevance of the HAP1 screening performed. For example, FANCM seems to interact with XRCC6 (Ku70) and PRKDC, two proteins associated with the NHEJ pathway. In addition, FANCM interacts with PALB2, RAD52, RAD511, BRCA1 and BRCA2, proteins associated with the HR pathway. Moreover, CDC16, SRSF1, CDT1 and MCM2 seem to interact with BRCA1, KIF4A with BRCA2 and RAD51, among other interactions with the HR and NHEJ repair pathway. PCBP1, SPC24, NUP88, NUP85, COPZ1, KLKL9 and PSMB4 although they do not seem to be associated directly with the selected HR and NHEJ proteins, they seem to be associated with other proteins found in the HAP1 screening which then interact with the NHEJ and HR pathways. Although an interaction of DDX28, CDK19, SASS6, MYBBP1A, THAP6, GABPB1, CRLF3 and MASTL with the HR and NHEJ pathways has not been detected using this software, it is possible that they also may interact with other proteins which were not included in the analysis or even with included proteins for which associations are unknown (FIG. 3).

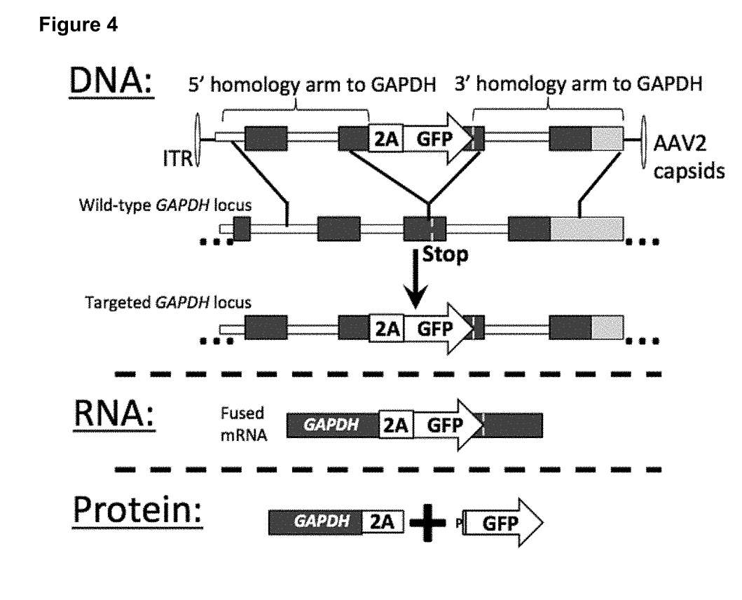

In order to begin validating the association of these proteins with the HR machinery, we began generating individual knockouts of each one of the selected 24 genes in wild type HAP1 cells. The CRISPR/Cas9 system was used to generate the knockouts after designing highly specific guide RNAs (sgRNAs) against exons 1, 2 or 3 of each gene. In addition, the AAV-GAPDH-2A-BLAS targeting construct was redesigned such that the BLAS transgene was replaced by the GFP coding sequence (new construct named AAV-GAPDH-2A-GFP) in order to allow for a precise quantification of target cells by FACs analysis (FIG. 4).

The first gene to be investigated in greater detail was FANCM. Three different knockouts were selected where the mutations generated by the CRISPR/Cas9 deletions caused frame-shifts in the FANCM gene reading-frame (clones B12: 32 bp del; C1:77 bp ins; C3:1 bp ins). The three FANCM knockout HAP1 cells as well as the original wild type HAP1 cell line were plated (1.4×10⁵ cells/well (24 well plate)) in duplicate and transduced with the AAV-GAPDH-2A-GFP targeting construct using an MOI: 5,000. Forty-eight hours post-transduction, cells were harvest for FACs analysis and an increase of approximately 10-fold in GFP-expressing cells was detected in the three knockout cell lines generated in relation to their wild type control (FIG. 5).

The FANCM gene is highly conserved since archea and belongs to the Fanconi anemia (FA) complementation group (FANC). FANC genes encode proteins belonging to the FA pathway, which becomes activated in response to breaks in single- and double-stranded DNA (e.g. DNA repair). Although mutations in many of the FA complementation groups have been associated with the Fanconi anemia recessive disorder, no disease has been linked to mutations in the FANCM gene, making its function less clear. In fact, FANCM is misnomer because initially a mutation in FANCM was found in a family with Fanconi Anemia but later it was found that their disorder was a consequence of mutations in a previously described Fanconi locus (FANCA). The data described here shows that when FANCM function is reduced, the efficiency of homologous recombination (HR) is enhanced.

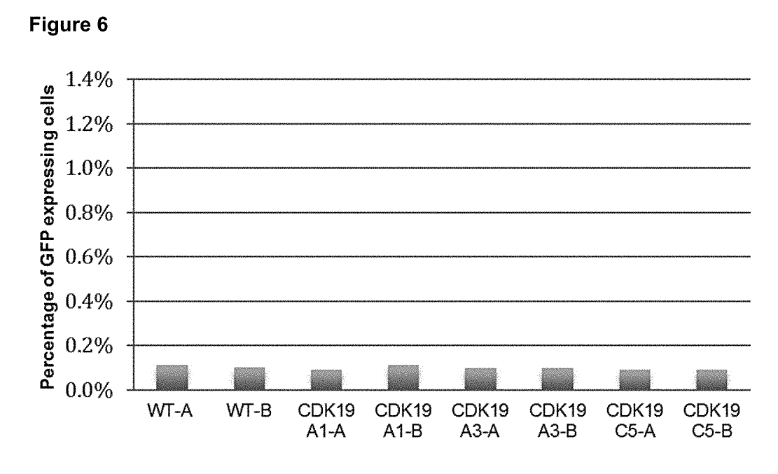

The second gene to be investigated in greater detail was the CDK19. Three knockouts were selected where the mutations generated by the CRISPR/Cas9 deletions caused frame-shifts in the CDK19 gene reading-frame (clones A1: 2 bp ins; A3:5 bp del; C5:280 bp del). The three CDK19 knockout HAP1 cells as well as the original wild type HAP1 cell line were plated (1.4×10⁵ cells/well (24 well plate)) in duplicate and transduced as described for FANCM knockout analysis. Forty-eight hours post-transduction, cells were harvest for FACs analysis and at this time no increase in the GFP-expressing cells was detected in relation to their wild type control (FIG. 6).

### Example 2

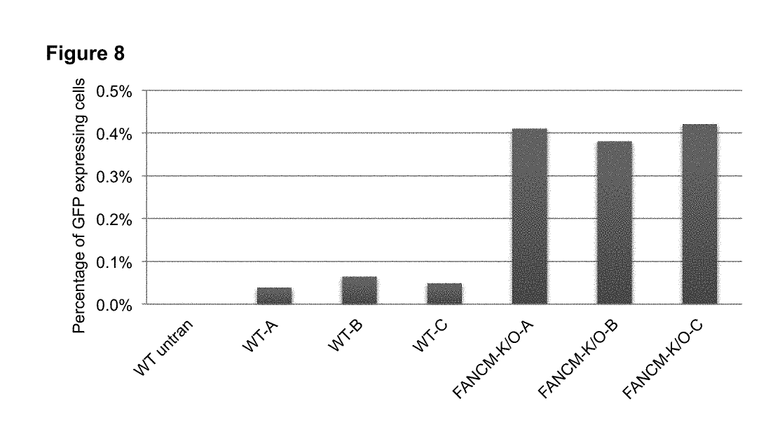

FANCM was knocked out in Hela cells after generating insertions and deletions (indels) in the gene by transfecting two different sgRNAs (Cas9 guide RNAs) against FANCM in association with the CRISPR/Cas9 system over two rounds of transfections (using the px459-puro plasmid). Forty-eight hours after the second transfection (Indels rate: >70%), cells were plated (0.6×10⁵ cells/well (24 well plate)) in triplicate and transduced with the AAV-GAPDH-2A-GFP targeting construct using an MOI: 51,000. Forty-eight hours post-transduction, cells were harvest for FACs analysis and an increase of approximately 10-fold in GFP-expressing cells was again detected in the FANCM knockout Hela cells in relation to their wild type control (FIG. 8).
FIG. 8. GFP expression in wild type and FANCM knock out HeLa cells transduced with AAV-GAPDH-2A-GFP. Cells were plated in triplicate 24 hours before being transduced with an MOI: 51,000. GFP expression was analyzed 48 hours post-transduction by FACs analysis. The letters A, B and C represent the triplicates.

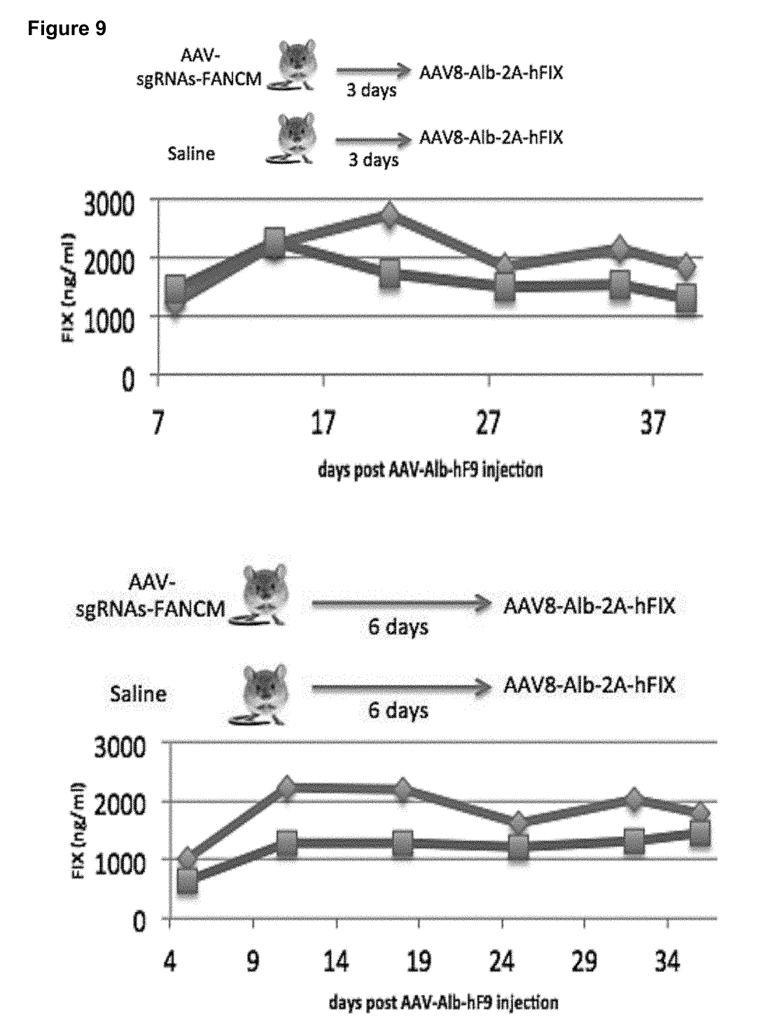

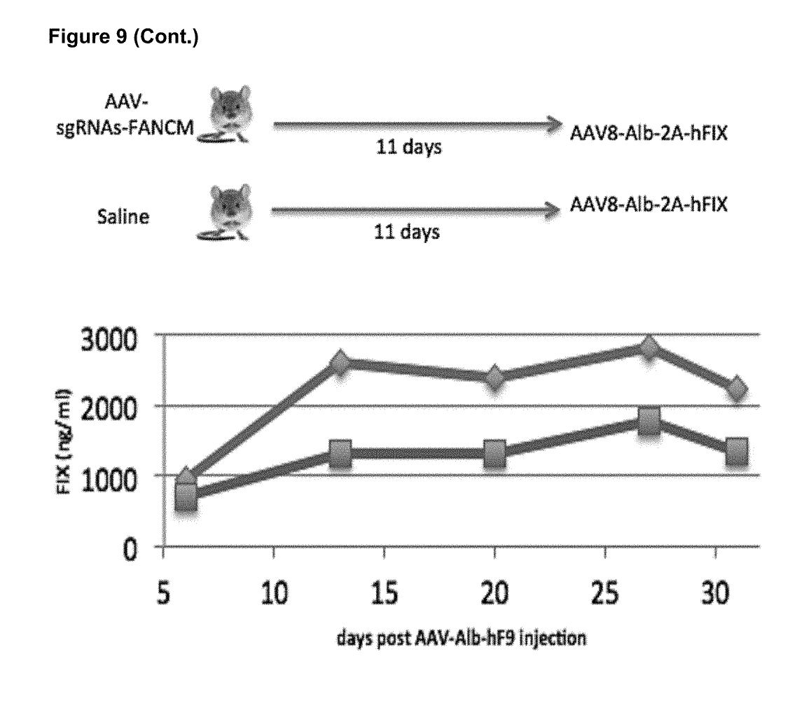

In addition, knockout of Fancm in a Cas9 mouse model also increased AAV mediated HR (FIG. 9) as detected by hFIX secretion (using the AAV-Albumin-2A-hF9 construct—FIG. 8). In these mice, tail-vein injection of an AAV-rh10 expressing sgRNAs against FANCM (1.8×10¹¹ vg) (diamonds) and saline (squares) was followed at various times by infusion of the AAV8-Albumin-2A-hF9 targeting vector (1×10¹² vg) which expresses human factor 9 upon precise integration into the Albumin locus, similar to the AAV-GAPDH-2A-GFP strategy. The relative levels of HR were determined by plasma human factor 9 measurements at different time points. The indel rate in the Fancm gene in these mice was >80%.
FIG. 9. Scheme of AAV injections for analysis of AAV-HR in vivo. Tail-vein injection of an AAV expressing sgRNAs against FANCM (diamonds) and saline (squares) was followed by infusion of the AAV8-Albumin-2A-hF9 targeting vector at various times. The relative levels of HR were determined by plasma human factor IX measurements.

### Example 3

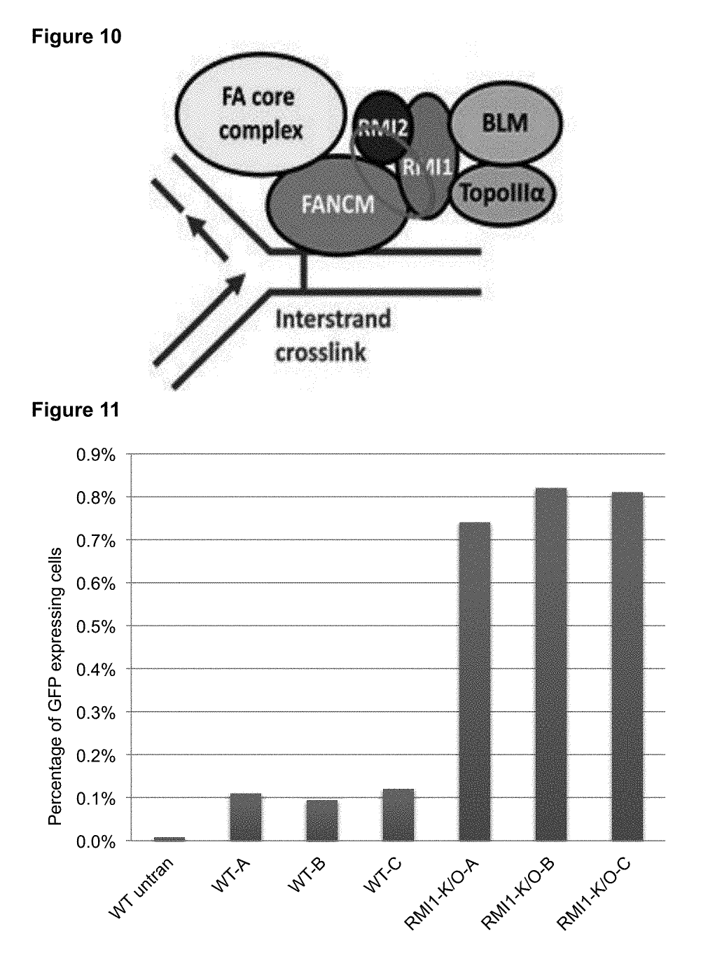

In order to test whether the knockout of other proteins associated with the FANCM complex can also increase HR, the RMI1 (RecQ-mediated genome instability 1) gene was selected. RMI1 anchors the Bloom dissolvasome complex to FANCM (FIG. 10), where impairment of this interaction can lead to an increase in sister chromatid exchange (SCE) events. Furthermore, RMI1 was identified in the genome-wide screening approach based on insertional mutagenesis in HAP1 cells (e.g., as described above).
FIG. 10. Scheme of FANCM protein interactions. When two replication forks have collided, FANCM recruits and interacts with two complexes of DNA repair: the FA core and the Bloom dissolvasome. The interaction between Bloom dissolvasome complex and FANCM occurs via the RMI subcomplex, which physically anchors the Bloom dissolvasome to FANCM by binding to a 34 amino acid motif in FANCM called MM2

The RMI1 gene was knocked out in Hela cells, similarly as performed above for the FANCM gene. The indel rate was >50%. As represented in FIG. 11, HR levels were also increased in these cells when RMI1 was knocked out, suggesting that the disruption of other proteins of the Bloom dissolvasome complex (e.g., Bloom DNA helicase (BLM), topoisomerase IIIα (Top3A, TopIIIα) and RecQ-mediated genome instability 2 (RMI2)) will increase the frequency of HR events.
FIG. 11. GFP expression in wild type and RMI1 knock out HeLa cells transduced with AAV-GAPDH-2A-GFP. Cells were plated in triplicate 24 hours before being transduced with an MOI: 37,000. GFP expression was analyzed 48 hours post-transduction by FACs analysis. The letters A, B and C represent the triplicates.

### Example 4

As shown above, cell lines containing knockouts of either FANCM or RMI1 have an increased level of homologous recombination (HR). In order to address whether the HR efficiency could be further increased when the two proteins are inhibit at the same time in a cell, both proteins were knocked down using siRNA. Another protein of the BTR complex (also known as the Bloom dissolvasome complex)—BLM (FIG. 1)—was also knocked down.

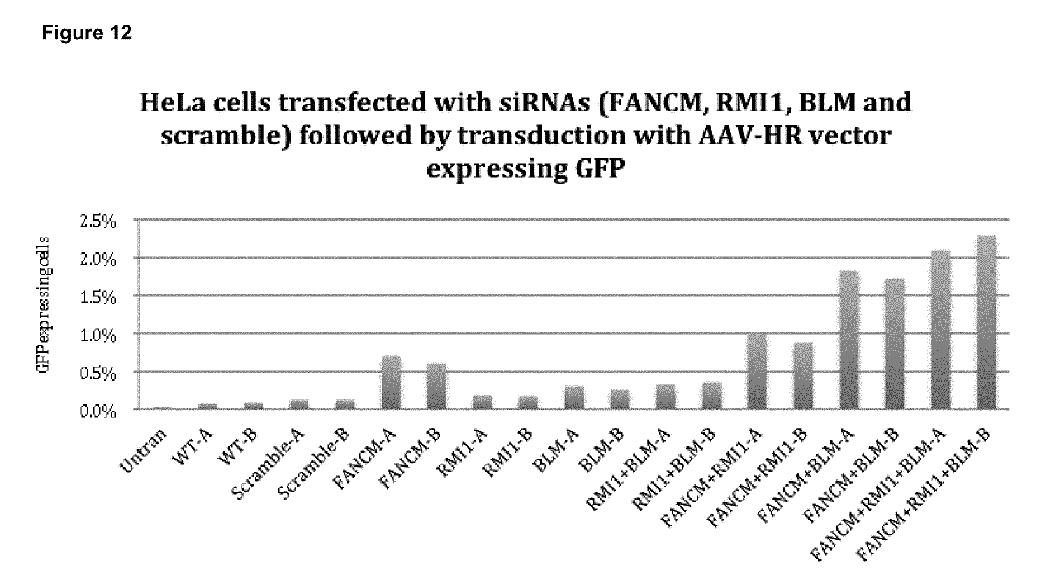

As shown in FIG. 12, the individual knockdown of all tested proteins increased HR levels as expected. Surprisingly, knockdown of FANCM with either RMI1 or BLM increased the levels of HR to higher levels. In addition, knockdown of all three proteins together further increased the HR levels: approximately 18 and 29-fold increase compared to cells transfected with a scramble siRNA and untransfected cells, respectively. Importantly, knockdown of RMI1 and BLM together did not increase the HR levels, suggesting that the increase in HR after inhibition of both FANCM and the BTR complex is independent and synergistic. Notably, both RMI1 and BLM proteins were also identified in the knockout HAP1 screen, reinforcing their true association with the increases in HR identified here. Therefore, the inhibition of both FANCM and proteins of the BTR complex (such as RMI1 and BLM) can be used in association to increase the efficiencies of AAV-mediated HR.
FIG. 12. GFP expression in wild type HeLa cells transfected with different combinations of siRNAs followed by transduction with AAV-GAPDH-2A-GFP. In order to perform this experiment, HeLa cells (0.4×10⁵ cells/well) were plated in duplicate in 24-wells plates and transfected in the next day with different siRNAs (Dharmacon) using Lipofectamine 3000 transfection reagent. A total of 250 ng (20 pmol) of each siRNA was used per well, totalizing 60 pmol when 3 different siRNAs were combined. siRNA scramble was used in the different transfection complexes in order to keep the same amount of siRNA transfected in all experiments. Seventy-two hours after transfection, cells were harvested, counted and re-plated (0.4×10⁵ cells/well) with AAV-GAPDH-2A-GFP (MOI: 51,000) for HR quantification by flow cytometry. GFP expression was analyzed 48 hours post-transduction by FACs analysis. The letters A and B represent the duplicates.

1. A method of inducing homologous recombination in a target cell, the method comprising introducing into the target cell: (a) a donor DNA, and (b) an agent that inhibits expression and/or function of an endogenous gene, or inhibits function of a gene product encoded by the endogenous gene, wherein the endogenous gene is selected from the group consisting of: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2, whereby a sequence of the donor DNA is incorporated into the target cell's genomic DNA.

2. (canceled)

3. The method of claim 1, wherein the endogenous gene is selected from the group consisting of: FANCM, DDX28, PCBP1, SFRS1/SRSF1, CDK19, DNAJA3/TID1, CDC16, and CDT1.

4. (canceled)

5. The method of claim 1, wherein the endogenous gene is selected from the group consisting of: FANCM, RMI1, BLM, TOP3A, and RMI2.

6. The method of claim 1, wherein the endogenous gene is FANCM or RMI1.

7. The method of claim 1, wherein the endogenous gene is FANCM.

8. The method of claim 1, wherein (b) is an agent or agents that inhibit expression and/or function of two or more endogenous genes, or inhibit function of gene products encoded by the two or more endogenous genes, wherein one endogenous gene is FANCM and another endogenous gene encodes a member of the BTR complex.

9. The method of claim 1, wherein said agent is an RNAi agent that specifically targets mRNA encoded by the endogenous gene.

10. The method of claim 1, wherein said agent is a genome editing agent that cleaves the target cell's genomic DNA at a locus encoding the endogenous gene.

11. The method of claim 10, wherein said genome editing agent is a CRISPR/Cas genome editing agent, a Zinc finger genome editing agent, or TALE genome editing agent.

12-14. (canceled)

15. The method of claim 1, wherein the method comprises cleavage of the target cell's genomic DNA with a gene editing endonuclease at a locus with homology to the donor DNA, prior to incorporation of the sequence of the donor DNA.

16-19. (canceled)

20. The method of claim 1, wherein the donor DNA is introduced into the cell by a virus.

21. The method of claim 20, wherein the virus is AAV.

22. A kit comprising (a) a donor DNA, and (b) an agent that inhibits expression and/or function of an endogenous gene, or inhibits function of a gene product encoded by the endogenous gene, wherein the endogenous gene is selected from the group consisting of: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2.

23. (canceled)

24. The kit of claim 22, wherein the endogenous gene is selected from the group consisting of: FANCM, DDX28, PCBP1, SFRS1/SRSF1, CDK19, DNAJA3/TID1, CDC16, and CDT1.

25. (canceled)

26. The kit of claim 22, wherein the endogenous gene is selected from the group consisting of: FANCM, RMI1, BLM, TOP3A, and RMI2.

27-28. (canceled)

29. The kit of claim 22, wherein said agent is an RNAi agent that specifically targets mRNA encoded by the endogenous gene.

30. The kit of claim 22, wherein said agent is a genome editing agent that cleaves the target cell's genomic DNA at a locus encoding the endogenous gene.

31-32. (canceled)

33. A method of inducing homologous recombination in a target cell, the method comprising introducing into the target cell: (a) a donor DNA, and (b) an agent that inhibits centriole assembly, whereby a sequence of the donor DNA is incorporated into the target cell's genomic DNA.

34. The method of claim 33, wherein the agent that inhibits centriole assembly is an inhibitor of Polo-like kinase 4 (Plk4).

35-45. (canceled)

46. A kit comprising (a) a donor DNA, and (b) an agent that inhibits centriole assembly.

47-49. (canceled)

50. A method of inducing homologous recombination in a mammalian target cell, the method comprising introducing a donor DNA into a mammalian target cell that has reduced expression and/or function of an endogenous gene, or has inhibited function of a gene product encoded by the endogenous gene, wherein the endogenous gene is selected from the group consisting of: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2, wherein said mammalian target cell comprises at least one of: (i) a loss-of-function mutation in the endogenous gene, and (ii) an RNAi agent, or DNA encoding said RNAi agent, that specifically targets mRNA encoded by the endogenous gene;
whereby a sequence of the donor DNA is incorporated into the target cell's genomic DNA.

51-53. (canceled)

54. The method of claim 50, wherein the endogenous gene is selected from the group consisting of: FANCM, RMI1, BLM, TOP3A, and RMI2.

55-56. (canceled)

57. The method of claim 50, wherein said agent is an RNAi agent that specifically targets mRNA encoded by the endogenous gene.

58. The method of claim 50, wherein said agent is a genome editing agent that cleaves the target cell's genomic DNA at a locus encoding the endogenous gene.

59-62. (canceled)

63. The method of claim 50, wherein the method comprises cleavage of the target cell's genomic DNA with a gene editing endonuclease at a locus with homology to the donor DNA, prior to incorporation of the sequence of the donor DNA.

64-76. (canceled)

77. A kit comprising (a) a genome editing agent, and (b) an agent that inhibits expression and/or function of an endogenous gene, or inhibits function of a gene product encoded by the endogenous gene, wherein the endogenous gene is selected from the group consisting of: FANCM, DDX28, PCBP1, SFRS1/SRSF1, CDK19, DNAJA3/TID1, CDC16, CDT1, SASS6, SPC24, CENPM, MCM2, KLHL9, MYBBP1A, NUP85, NUP88, KIF4A, MASTL, CRLF3, SET, GABPB1, PSMB4, THAP6, COPZ1, RMI1, BLM, TOP3A, and RMI2.

78. (canceled)

79. The kit of claim 77, wherein the endogenous gene is selected from the group consisting of: FANCM, DDX28, PCBP1, SFRS1/SRSF1, CDK19, DNAJA3/TID1, CDC16, and CDT1.

80. (canceled)

81. The kit of claim 77, wherein the endogenous gene is selected from the group consisting of: FANCM, RMI1, BLM, TOP3A, and RMI2.

82-87. (canceled)
